(19) United States
(12) Patent Application Publication
(10) Pub. No.: US 2005/0123553 A1
(43) Pub. Date: Jun. 9, 2005

(54) Amyloid beta-peptide and methods of use

(76) Inventors: Alon Monsonego, Moshav Nir-Banim (IL); Howard Weiner, Brookline, MA (US); Dennis J. Selkoe, Jamaica Plain, MA (US)

Correspondence Address: FISH & RICHARDSON PC, 225 FRANKLIN ST, BOSTON, MA, 02110, US

(21) Appl. No.: 10/903,279

(22) Filed: Jul. 30, 2004

Related U.S. Application Data
(60) Provisional application No. 60/491,485, filed on Jul. 30, 2003.

(51) Int. Cl. G01N 33/53; G01N 33/567; A61K 39/00; C07K 14/47

(52) U.S. Cl. 424/185.1; 514/12; 530/350; 435/7.2

(57) ABSTRACT

Novel compositions containing an amyloid-β peptide (Aβ) and methods of using these compositions for treating and preventing Aβ protein related (e.g., an amyloid fibril) disorders such as Alzheimer's disease are described herein. Also described herein are methods for identifying a vaccine, and methods of vaccinating a patient with an Aβ peptide composition identified using the methods described herein.

### CROSS-REFERENCE TO RELATED APPLICATIONS

This application claims priority to U.S. Patent Application No. 60/491,485, filed Jul. 30, 2003, the contents of which is incorporated by reference in its entirety.

### GOVERNMENT SUPPORT

The work described herein was supported, at least in part, by grants from the National Institutes of Health. The United States Government may therefore have certain rights in this invention.

### TECHNICAL FIELD

This invention relates to novel compositions and methods for treating and preventing amyloid-β (Aβ) protein related disorders such as Alzheimer's disease.

### BACKGROUND

Alzheimer's disease (AD), first described by the Bavarian psychiatrist Alos Alzheimer in 1907, is a progressive neurological disorder that begins with short-term memory loss and proceeds to disorientation, impairment of judgment and reasoning and, ultimately, dementia. The course of the disease usually leads to death in a severely debilitated, immobile state between four and 12 years after onset. Approximately 4 million Americans have Alzheimer's disease. One in 10 persons over 65, and nearly half of those over 85, have Alzheimer's disease. Alzheimer's disease costs U.S. society at least $100 billion a year, with neither Medicare nor most private health insurances covering the long-term care most patients need.

Alzheimer's disease is characterized by the progressive accumulation of the amyloid-β(Aβ) protein in limbic and association cortices, where some of it precipitates to form a range of amorphous and compacted (fibrillar) extracellular plaques (Price, D. L., and Sisodia, S. S. Annu Rev Neurosci 21:479-505, 1998; Selkoe, D. J. Physiol Rev 81:741-766, 2001). These plaques, particularly the more compacted ones, are associated with dystrophic neurites (altered axons and dendrites) (Urbanc, B., et al., Proc Natl Acad Sci USA 99:13990-13995,2002.), activated microglia and reactive astrocytes. Cleavage of the amyloid precursor protein (APP) by the β- and γ-secretases releases both the Aβ1-40 and Aβ1-42 peptides, the latter being more prone to aggregation and induction of neurotoxicity (Jarrett, J. T., and Lansbury, P. T., Jr., Cell 73:1055-1058, 1993.). Aβ1-42 has the following sequence: DAEFRHDSGYEVHHQKLVFFAEDVGSNKGAIIGLMVGGVVIA (SEQ ID NO:1). The amyloid precursor protein (APP) is 770 amino acids in length and has the following sequence:

| | | |
|---|---|---|
| mlpqlallll aawtaralev ptdgnaglla epqiamfcgr lnmhmnvqng kwdsdpsgtk | 61 | (SEQ ID NO:2) |
| tcidtkegil qycqevypel qitnvveanq pvtiqnwckr grkqckthph fvipyrclvg | 121 | |
| efvsdallvp dkckflhqer mdvcethlhw htvaketcse kstnlhdygm llpcgidkfr | 181 | |
| gvefvccpla eesdnvdsad aeeddsdvww ggadtdyadg sedkvvevae eeevaeveee | 241 | |
| eadddedded qdeveeeaee pyeeatertt siattttttt esveevvrev cseqaetgpc | 301 | |
| ramisrwyfd vtegkcapff yggcggnrnn fdteeycmav cqsamsqsll kttqeplard | 361 | |
| pvklpttaas tpdavdkyle tpgdenehah fqkakerlea khrermsqvm reweeaerqa | 421 | |
| knlpkadkka viqhfqekve sleqeaaner qqlvethmar veamlndrrr lalenyital | 481 | |
| qavpprprhv fnmlkkyvra eqkdrqhtlk hfehvrmvdp kkaaqirsqv mthlrviyer | 541 | |
| mnqslsllyn vpavaeeiqd evdellqkeq nysddvlanm iseprisygn dalmpsltet | 601 | |
| kttvellpvn gefslddlqp whsfgadsvp antenevepv darpaadrql ttrpgsgltn | 661 | |
| ikteeisevk mdaefrhdsg yevhhqklvf faedvgsnkg aiiglmvggv viatvivitl | 721 | |
| vmlkkkqyts ihhgvvevda avtpeerhls kmqqngyenp tykffeqmqn | | |

Although Alzheimer's disease is associated with local innate immune responses, the induction of systemic adaptive immune responses to Aβ in mouse models of Alzheimer's disease has been found to be beneficial for both the neuropathological and behavioral changes that these mice develop (Schenk, D. et al. Nature 400:173-177, 1999; Morgan, D. et al. Nature 408:982-985, 2000; Janus, C. et al. Nature 408:979-982, 2000; Weiner, H. L., et al., Ann Neurol 48:567-579, 2000; Lemere, C. A. et al., Ann N Y Acad Sci 920:328-331, 2000; Bard, F. et al. Nat Med 6:916-919, 2000; Dodart, J. C. et al. Nat Neurosci 5:452-457, 2002). However, a human clinical trial in which an Aβ1-42 synthetic peptide was administered parenterally with the adjuvant QS21 to patients with Alzheimer's disease was discontinued when Approximately 5% of 300 treated patients developed what appeared to be a self-limited aseptic meningoencephalitis. One hypothetical cause of this reaction was an immune response to Aβ (Hock, C. et al., Nat Med 8:1270-1275, 2002; Pfeifer, M. et al., Science 298:1379, 2002; Nicoll, J. A. et al., Nat Med 9:448-452, 2003).

### SUMMARY

Various methods for treating, preventing, or reducing the incidence or severity of Alzheimer's disease and other disorders related to the accumulation of extracellular Aβ protein-containing plaques (e.g., an amyloid fibril disorder) are described. The methods include administering to a patient a peptide or a composition that elicits a T-cell response that is beneficial. For example the T-cell response can reduce Aβ plaques in the brain of a subject, without causing an encephalitic response. In some instances, the methods include administering to a patient a composition that elicits a Th2 response (i.e., Th2 reactive T-cells) to an Aβ peptide, e.g., a peptide consisting of all or a portion of SEQ ID NO:1. Certain peptides and compositions elicit a Th2 response that is stronger than a Th1 response, e.g., the composition includes a greater increase in peptides having a Th2 epitope than peptides having a Th1 epitope. Also disclosed are methods of identifying peptides and compositions useful for eliciting a T-cell response to Aβ protein, such as a Th2 response, and peptides and compositions for use in the treatment and prevention of Alzheimer's disease and other amyloid fibril disorders. It can be desirable to identify an immunogenic composition that elicits an effective T-cell response, such as a Th2 immune response, for a selected individual, as not all individuals respond in the same manner to a given immunogenic composition. For example, the response of an individual can vary depending on the MHC Class II allele presented. Methods for monitoring a patient's immune response, e.g., a response to a vaccine, are also described. Accordingly, methods are described that provide for the identification of a specific treatment regime that will most effectively meet the needs of an individual patient.

In one aspect, the invention features a peptide including at least 12 contiguous amino acids of SEQ ID NO:1, excluding amino acids 1-5, (e.g., 1-6, 1-7, 1-8, 1-9, 1-10, 1-11, 1-12, 1-13, or 1-14), wherein the peptide elicits T-cell response upon administration to a subject. The T-cell response can be determined in a variety of ways, including by exposing the peptide to PBMCs. In some instances, the T-cell response is in the central nervous system, for example in the brain.

In some embodiments, the T-cell response includes a reduction of AD plaques, for example, via clearance of Aβ with microglia or macrophages.

In some embodiments, the T-cell response includes an increase in Th2 Aβ reactive T-cells. In other embodiments, the T-cell response includes an increase in Th1 Aβ reactive T-cells. In still other embodiments, the T-cell response includes an increase in both Th1 Aβ reactive T-cells and Th2 Aβ reactive T-cells.

In some embodiments, the peptide includes 12 contiguous amino acids from amino acids 15-34 of SEQ ID NO:1, e.g., amino acids 19-33 or 30-42. In some embodiments, the peptide includes at least 15 contiguous amino acids, for example, the peptide can include amino acids 15-30, 16-31, 17-32, 18-33, 19-34, 20-35, 21-36, 22-37, 23-28, 24-29,25-40, 26-41, or 27-42 of SEQ ID NO:1. In some embodiments, the peptide includes a T-cell epitope, a Th2 epitope, or a Th1 epitope.

In another aspect, the invention features a composition including a peptide described herein. The composition can include an adjuvant, for example alum, incomplete Freud's adjuvant, complete Freud's adjuvant, or QS21.

In another aspect, the invention features a peptide including at least 12 contiguous amino acids of SEQ ID NO:1, for example amino acids 30-42, and an adjuvant, wherein the composition elicits an increase an Th1 Aβ reactive T-cells that is less than ten times as great as an increase in Th2 Aβ reactive T-cells when exposed to PBMCs. In some embodiments, the composition elicits a greater increase in Th2 Aβ reactive T-cells than an increase in Th1 Aβ reactive T-cells when exposed to PBMCs. In some instances, the peptide can include amino acids 15-42 of SEQ ID NO:1. In other instances, the peptide includes at least 15 contiguous amino acids of SEQ ID NO:1. For example, the peptide can include amino acids 15-30, 16-31, 17-32, 18-33, 19-34, 20-35, 21-36, 22-37, 23-28, 24-29, 25-40, 26-41, or 27-42 of SEQ ID NO:1. The peptide can include a Th2 epitope or a Th1 epitope. In some embodiments the peptide does not include amino acids 1-14 of SEQ ID NO:1.

In some embodiments, the adjuvant includes alum or incomplete Freud's adjuvant. In some embodiments, the composition includes amino acids 18-33 of SEQ ID NO:1 and the adjuvant includes alum.

In some embodiments, the composition can include additional elements. For example, the composition can also include a pharmaceutically acceptable carrier.

The relative increase in Th2 Aβ reactive T-cells versus increase in Th1 Aβ reactive T-cells can vary depending on the composition. The relative immune responses can be characterized in the following ranges: for example, the increase in Th2 Aβ reactive T-cells can be three to five, five to eight, eight to ten, or more than ten times greater than the increase in Th1 Aβ reactive T-cells.

The relative Th2 and Th1 immune responses can be measured in a variety of ways. For example, the amount of Th2 Aβ reactive T-cells can be determined by expression of IL-4, IL-5, IL-10, or IL-13; and the amount Th1 Aβ reactive T-cells can be determined by expression of IFN-γ, IL-12, or TNF-α. Alternatively the amount of Th2 Aβ reactive T-cells can be determined by the expression of IgG1 or IgG4 and the amount Th1 Aβ reactive T-cells can be determined by expression of IgG2 or IgG3.

Various factors can influence the immune response of a composition. For example, in some instances, the peptide elicits a greater increase in Th2 Aβ reactive T-cells than increase in Th1 Aβ reactive T-cells in the absence of adjuvant. In other instances, the adjuvant elicits a greater increase in Th2 Aβ reactive T-cells than increase in Th1 Aβ reactive T-cells in the absence of peptide. In still other instances both the peptide and the adjuvant independently a greater increase in Th2 Aβ reactive T-cells than increase in Th1 Aβ reactive T-cells. The increase in a Th2 response, due to any elements present in the composition independently, can be three to five, five to eight, eight to ten, or more than ten times greater than the increase in Th1 response. In some instances, the method of administration of the composition can influence the relative increase in Th2 response. For example, in some instances, nasal administration of the composition can influence the relative increase in Th2 response compared to the increase in Th1 response.

In another aspect, the invention features a method for eliciting an immune response in a patient, such as a T-cell immune response in the central nervous system. The method includes administering to the patient any of the peptides or compositions described herein. In some embodiments, the immune response includes a reduction of Aβ plaques in the central nervous system, for example the brain. The reduction of Aβ plaques can occur, for example, by the clearance of Aβ via microglia or macrophages. In some embodiments, the immune response is a T-cell response. For example, in some embodiments, the peptides or compositions elicit an increase in Th2 Aβ reactive T-cells in the patient. In some embodiments, the peptides or compositions elicit an increase in Th1 Aβ reactive T-cells in the patient. In still other embodiments, the peptides or compositions elicit an increase in Th1 Aβ reactive T-cells and an increase in Th2 Aβ reactive T-cells in the patient.

The peptides and compositions can be administered in a variety of manners. For example, the compound can be administered orally, parenterally, or mucosally (e.g., nasally).

In another aspect the invention features a method of treating a patient having an amyloid fibril disorder. The method includes administering to the patient any of the peptides or compositions described herein. The amyloid fibril disorder can include a form of dimentia (e.g., Alzheimer's disease). Other amyloid fibril disorders amenable to treatment by the methods described herein include Down's Syndrome, Dutch Type Hereditary Cerebral Hemorrhage Amyloidosis, Reactive Amyloidosis, Familial Mediterranean Fever, Familial Amyloid Nephropathy with Urticaria and Deafness, Muckle-Wells Syndrome, Idiopathic Myeloma; Macroglobulinemia-Associated Myeloma, Familial Amyloid Polyneuropathy, Familial Amyloid Cardiomyopathy, Isolated Cardiac Amyloid, Systemic Senile Amyloidosis, Adult Onset Diabetes, Insulinoma, Isolated Atrial Amyloid, Medullary Carcinoma of the Thyroid, Familial Amyloidosis, Hereditary Cerebral Hemorrhage with Amyloidosis, Familial Amyloidotic Polyneuropathy, Scrapie, Creutzfeldt-Jacob Disease, Gerstmann Straussler-Scheinker Syndrome, Bovine Spongiform Encephalitis, a Prion-mediated disease, or Huntington's Disease.

In another aspect, the invention features a method of immunizing a patient by administering to the patient any of the peptides or compositions described herein. Any suitable route of administration can be used.

In one embodiment, a method of immunizing a patient includes identifying a patient as free of infection in the central nervous system, and administering to the identified patient a composition including a peptide including at least 12 contiguous amino acids of SEQ ID NO:1. In some instances, patient is identified as free of infection in the central nervous system by being substantially free of INF-γ in the central nervous system, for example by taking a sample of cerebral spinal fluid and measuring for the presence of INF-γ using an ELISA assay. In another embodiment, the method also includes isolating PBMCs from a patient; exposing the PBMCs to a library of Aβ peptides; and identifying an Aβ peptide within the library that elicits a greater increase in Th2 Aβ reactive T-cells than an increase in Th1 Aβ reactive T-cells when exposed to PBMCs.

In another embodiment, a method of identifying a peptide for eliciting an immune response in an individual includes isolating PBMCs from a patient; exposing the PBMCs to a library of Aβ peptides; and identifying an amyloid-β peptide within the library that elicits a T-cell immune response when exposed to PBMCs.

In another embodiment, the invention includes a method of immunizing an individual including isolating PBMCs from an individual; exposing the PBMCs to a library of Aβ peptides; identifying an Aβ peptide within the library that elicits a T-cell response; and administering to the individual the identified Aβ peptide, thereby immunizing the individual. The method can also include determining the relative amounts of Th2 Aβ reactive T-cells and Th1 Aβ reactive T-cells at a time prior to administering to the individual the identified Aβ peptide, t₀, and determining the relative amounts of Th2 Aβ reactive T-cells and Th1 Aβ reactive T-cells at a time after administering to the individual the identified Aβ peptide, t₁.

In another embodiment, the invention includes a method of identifying a candidate composition for eliciting an immune response in an individual. The method includes isolating PBMCs from a patient; exposing the PBMCs to a library of compositions, each composition including an Aβ peptide including at least 5 (e.g., 6, 7, 8, 9, 10, 11, 12, etc.) contiguous amino acids of SEQ ID NO:1 and an adjuvant; and identifying as the candidate composition a composition within the library of compositions that elicits a T-cell response.

In another aspect, the invention features a method of immunizing an individual, where the method includes: isolating PBMCs from a patient; exposing the PBMCs to a library of compositions, each composition including an Aβ peptide including at least 5 (e.g., 6, 7, 8, 9, 10, 11, 12, etc.) contiguous amino acids of SEQ ID NO:1 and an adjuvant; identifying a composition within the library that elicits a T-cell response; and administering the identified composition to the individual, thereby immunizing the individual. In some embodiments, the method also includes determining the relative amounts of Th2 Aβ reactive T-cells and Th1 Aβ reactive T-cells at a time prior to administering to the individual the identified Aβ peptide, t₀, and determining the relative amounts of Th2 Aβ reactive T-cells and Th1 Aβ reactive T-cells at a time after administering to the individual the identified Aβ peptide, t₁.

In another aspect, the invention includes a method for determining an adverse reaction to an immunization with an Aβ antigen in a patient. The method includes measuring the relative amounts of Th2 Aβ reactive T-cells and Th1 Aβ reactive T-cells in a patient at an initial time prior to immunization, t₀; immunizing the patient with an Aβ antigen; measuring the relative amounts of Th2 Aβ reactive T-cells and Th1 Aβ reactive T-cells in a patient at a time subsequent to immunization, t₁; and comparing the relative responses of Th2 Aβ reactive T-cells and Th1 Aβ reactive T-cells at t₀ and t₁, wherein an increase in relative amounts of Th1 Aβ reactive T-cells to relative amounts of Th2 Aβ reactive T-cells from t₀ to t₁ indicates an adverse reaction to the immunization.

In another aspect, the invention includes a method for determining an adverse reaction to an immunization with an Aβ antigen in a patient. The method includes measuring an amount of Th1 Aβ reactive T-cells and an amount of Th2 Aβ reactive T-cells in a patient at an initial time prior to immunization, t₀; immunizing the patient with an Aβ antigen; measuring an amount of Th1 Aβ reactive T-cells and an amount of Th2 Aβ reactive T-cells in a patient at a time subsequent to immunization, t₁; determining whether the patient experienced an increase in Th1 Aβ reactive T-cells that is more than ten times greater than the increase in Th2 Aβ reactive T-cells from t₀ to t₁ and whether INF-γ is present in the central nervous system, wherein an relative increase in Th1 Aβ reactive T-cells greater than ten times the increase in Th2 Aβ reactive T-cells from t₀ to t₁ combined with the presence of INF-γ in the central nervous system indicates an adverse reaction.

In another aspect, a method of diagnosing an Aβ fibril disorder in a patient is described. The method includes measuring the amount of Aβ reactive T-cells in a patient; comparing the amount Aβ reactive T-cells to a standard; and diagnosing the patient with an Aβ fibril disorder if the amount of Aβ reactive T-cells in the patient is higher than the standard.

For the methods described above, the library of Aβ peptides can include, for example, peptides including at least 5 (e.g., at least 6, 7, 8, 9, 10, 11, 12, 13, 14, or 15) contiguous amino acids of SEQ ID NO:1. The T-cell response can be any of the T-cell responses described herein, and the T-cell response can be measured using any of the methods described herein. In some embodiments, the Aβ peptides are attached (e.g., covalently or non-covalently) to an MHC class II molecule.

The terms “peptides”, “proteins”, and “polypeptides” are used interchangeably herein.

The term “Th” refers to a helper T cell. Multiple types of T cell epitopes exist, each of which corresponds to a unique immune response.

The term “MHC Class II molecule” refers to an antigen-presenting molecule found primarily on dendritic cells, the best antigen presenting cells, and also on macrophages and B lymphocytes.

The term “HLA antigen” refers to an MHC class II molecule in human.

The term “PBMCs” refers to peripheral blood mononuclear cells.

The term “epitope” refers to a particular site within a biomolecule (e.g., an antigen) to which an antibody binds.

The term “library”, as used herein, refers to a collection of elements, such as peptides or compositions, which can be used to rapidly assay a biological property (e.g., an immune response) of a large number of samples simultaneously. The library can be unordered; for example, the library can be a mixture of elements. Alternatively, the library can be ordered or arrayed, such that each element within the library is individually entered into an order, for example into a physical position of a two-dimensional array.

A “treatment”, as used herein, includes any therapeutic treatment, e.g., the administration of a therapeutic agent or substance, e.g., a drug.

The term “immune response” refers to the development of a humoral (antibody mediated) and/or a cellular (mediated by antigen-specific T cells or their secretion products) response directed against an antigen in a vertebrate individual. Such a response can be an active response induced by administration of immunogen or a passive response induced by administration of antibody or primed T-cells or B cells which can act as antigen presenting cells. A cellular immune response is elicited by the presentation of polypeptide epitopes in association with Class I or Class II MEC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by standard proliferation assays (CD4⁺ T cells) or CTL (cytotoxic T lymphocyte) assays known in the art. The relative contributions of humoral and cellular responses to the protective or therapeutic effect of an immunogen can be distinguished by separately isolating immunoglobulin (IgG) and T-cell fractions from an immunized syngeneic animal and measuring protective or therapeutic effect in a second subject.

The term “adjuvant” refers to a substance that is added to a vaccine to improve the immune response. In some instances, the adjuvant can result in a lower necessary dose of vaccine required to produce sufficient quantity of antibodies. Such adjuvants can work by speeding the division of lymphocytes and by keeping the antigen in the area where the immune response is taking place. Some examples of adjuvants include alum, aluminum phosphate, aluminum hydroxide gel, and Freund's adjuvant (complete or incomplete).

The term “pharmaceutically acceptable carrier” refers to a carrier that can be administered to a patient together with a composition of this invention. The carrier does not destroy the pharmacological activity of the composition and is nontoxic when administered in doses sufficient to deliver a therapeutic amount of the composition.

The term “effective amount” refers to the amount of a composition required to be administered to a patient that induces a desired response. Some examples of effective amounts include the amount of a pharmaceutical composition required to alleviate a symptom such as pain or inflammation, or the amount of a composition required to induce an immune response in a patient, e.g., a vaccine. An effective amount can be determined using objective factors such as a measurable reduction in inflammation, or an effective amount can be measured subjectively, for example, based on a patient's description of a change in a symptom such as pain.

The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts the immunogenicity of Aβ 1-42 relative to Aβ 1-40.

FIGS. 2A and 2B depict a comparison of Aβ T-cell reactivity in adult patients, elderly patients, and elderly patients with Alzheimer's disease.

FIGS. 3a-3o depict epitope specificity and cytokine profiles of Aβ reactive T-cell lines.

FIGS. 4A-4C depict exhibition of Th1, Th2, and Th0 phenotypes in activated Aβ-reactive T-cells after stimulation with autologous PBMCs and Aβ peptides.

FIGS. 5A-D depict Aβ 1-42 specific T-cell line (DRB1 1501/1101) responses to various Aβ epitopes.

FIGS. 6A-D depict a comparison of immune response for various immunogenic epitopes in mice.

FIGS. 7A-E depict the enecphalitic response of APP/TFN-γ double Tg mice to the T-cell epitope Aβ10-24.

FIGS. 8A-C depicts T-cell response in APP/IFN-γ mice upon immunization with Aβ10-24.

FIGS. 9A-B depict a decrease in Aβ burden in the hippocampus regions associated with activated microglia/macrophages in APP/IFN-g mice immunized with Aβ10-24.

FIGS. 10A-B and 11A-C depict the enhanced uptake of Aβ by microglia upon the treatement of microglial cultures with IFN-γ.

### DETAILED DESCRIPTION

The invention is based, in part, on the discovery of conditions which elicit T-cell responses directed to Aβ plaques in the central nervous system (CNS), which have the potential effect of clearing pathogenic forms of Aβ. It was further discovered that Aβ can become an encephalitogenic antigen when three conditions are satisfied: 1. when Aβ is accumulated and deposited in the brain, as occurs with age; 2. when the genetic background of the individual predisposes to T cell response to a high affinity Aβ T-cell epitope; and 3. when there is a pro-inflammatory signal such as IFN-γ.

Described below are studies that demonstrate varied immunogenic responses in mice to Aβ. Further studies suggest that the degree of immunogenicity can result from affinity of an epitope elected by the IA^{b} MHC class II rather than mechanisms of T-cell selection in the thymus, demonstrating that the choice of MHC class II alleles can be important for the strength and phenotype of adaptive immune response evoked to Aβ following immunization. Moreover, it was demonstrated that IFN-γ facilitates microglia motility and uptake of Aβ as well as T-cell motility and synapse formation, which can promote the T-cell responses in the brain.

The invention is further based, in part, on the discovery that circulating Aβ-reactive T cells are present in patients with Alzheimer's disease and increase with aging and on the identification in patients of DR-restricted T-cell epitopes reactive with Aβ.

The invention is further based, in part, on the discovery that circulating Aβ-reactive T cells are present in patients with Alzheimer's disease and increase with aging and on the identification in patients of DR-restricted T-cell epitopes reactive with Aβ. These findings were unexpected given that aging is generally associated with increased susceptibility to infection agents and defective T-cell priming (Linton, P. J., et al., J Exp Med 184:1891-1900, 1996; Bansal-Pakala, P., and Croft, M., J Immunol 169:5005-5009, 2002) a trend also observed in subjects when cells were stimulated with anti-CD3/CD28 or glatiramer acetate.

Moreover, Th1-type immune responses have been implicated in many autoimmune disorders, wherein Th2-type responses have been shown to inhibit autoimmune disease. Accordingly, a desirable vaccine or treatment for Alzheimer's disease and other Aβ protein-related disorders would include a composition that selectively elicits a Th2 response over a Th1 response (i.e., elicits a greater increase in Th2 Aβ reactive T-cells than an increase in Th1 Aβ reactive T-cells). Factors that influence which immune response is elicited include: the antigen epitope, the adjuvant, and the method of administration of the immunogenic composition.

Described below are studies showing that certain Aβ peptides alone, or in combination with a specific adjuvant, can elicit a Th2 response. For example, Aβ1-42, Aβ15-42, and Aβ16-30 elicit a Th2 response. Moreover, certain adjuvants can elicit a selective Th2 response, e.g., alum. Nasal administration of representative peptide compositions also elicits a Th2 response. These peptides can be use as components of vaccines against or treatments for Alzheimer's disease or other amyloid fibril related disorders. In some cases, it can be desirable to test an individual to determine which portion of Aβ will elicit an effective Th2 response in order to provide an effective vaccine or treatment for that individual.

Detection of an Endogenous T-Cell Responses to Aβ in a Patient.

T-cell responses to Aβ were measured using a split-well assay that has been successful in demonstrating reactivity to self antigens (Fukaura, H., et al., J Clin Invest 98:70-77 (1996)). FIG. 12 (Table 1) shows an example of the results of a split-well assay performed with PBMCs isolated from a healthy adult using 40 μg/ml Aβ1-42. In this individual, 13.3% (4/30) of the wells demonstrated Aβ-mediated proliferation, with 20%, 14%, and 20% positive wells, respectively. At these lower antigen concentrations, similar numbers of positive wells and similar SIs were obtained, with no wells having an SI≦0.5 (not shown), the latter is possibly to be secondary to activation-induced T-cell death. Because Aβ-reactive T-cell lines usually proliferated maximally with 10 μg/ml Aβ1-42, split-well assays were performed using 10 μg/ml Aβ1-42 with the test subjects.

Aβ1-42 is More Immunogenic than Aβ1-40.

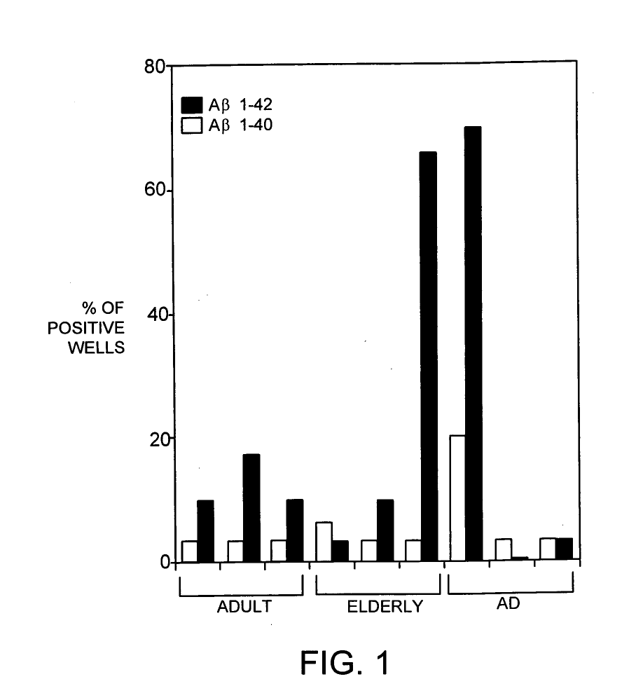

Prior to testing large numbers of subjects using the split-well assay, the relative antigenicity of the two major forms of AD protein, Aβ1-40 and Aβ1-42 was determined. Split-well assays were performed in 9 subjects [3 young adults (25-40 yr old), 3 older healthy subjects (50-85 yr old) and 3 patients with Alzheimer's disease (58-90 yr old)] using either Aβ1-40 or Aβ1-42. Of these, 6 subjects (3 adults, 1 older individual, and 2 Alzheimer's disease patients) showed a markedly higher percentage of positive wells when Aβ1-42 was used as the antigen, whereas only 3 subjects, who had low percentages of positive wells, showed similar or slightly higher reactivity evoked by Aβ1-40 (see FIG. 1). Based on these results, we chose to measure T-cell responses to Aβ1-42 exclusively.

Increased T-Cell Reactivity to Aβ is Shown in Elderly Subjects and Patients with Alzheimer's Disease.

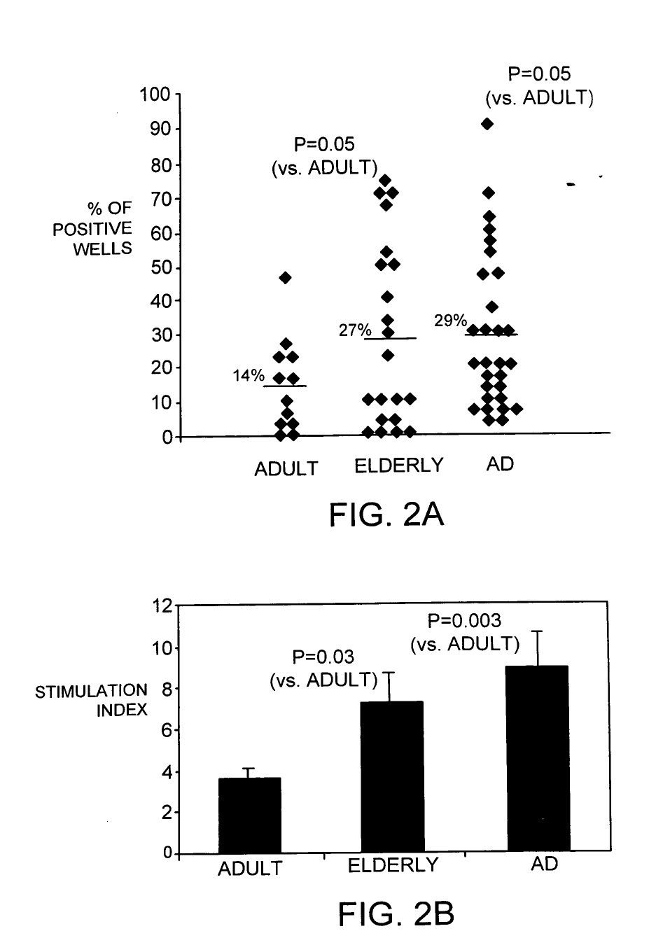

Split-well assays were performed on PBMCs derived from young adults (25-40 yr old, n=13), older healthy subjects (50-85 yr old, n=22) and patients with Alzheimer's disease (58-90 yr old, n=29), using Aβ1-42 (10 ug/ml) as the stimulating antigen. The percentage of positive T-cell reactivity to Aβ1-42 (as defined by a stimulation index of >2.5 and a Δcpm≧2,000) was significantly higher in healthy elderly subjects (mean 27%) than normal adult subjects (mean 14%) (p=0.05) (see FIG. 2A). Regression analysis demonstrated a trend linking increased T-cell reactivity with age (r=0.29, p=0.09). In addition, a significantly higher average of percent positive wells (29%) was also observed in the group of patients with Alzheimer's disease (different from adults at p=0.01), whereas the elderly group did not differ from the Alzheimer's disease group (see FIG. 2A). Furthermore, several of the elderly healthy and Alzheimer's disease subjects had a markedly elevated percentage of positive wells (>50%) (see FIG. 2A). Although the difference between the mean reactivities of the Alzheimer's disease and elderly subjects was not significant, 23% (5 of 22) of the elderly had no T-cell reactivity to Aβ, whereas this was not observed in patients with Alzheimer's disease (0 of 29) (p=0.03).

While the percent positive wells provides a measure of reactivity to Aβ, it does not assess the magnitude of the response. Accordingly, to determine whether the strength of the response to Aβ as measured by mean SIs was also higher in the elderly and Alzheimer's disease subjects, average SIs were calculated from the positive wells of each of the subjects tested. Based on these calculations, average SIs were significantly higher in the healthy elderly and Alzheimer's disease groups than in the adult group (p=0.03 and 0.003, respectively) (see FIG. 2B).

The increased reactivity to Aβ was significant, given that T-cell immunity tends to decrease with aging (Linton, P. J. et al., J Exp Med 184:1891-1900, 1996; Bansal-Pakala, P., and Croft, M., J Immunol 169:5005-5009, 2002) as older humans are more susceptible to infectious diseases and the effects of vaccination are reduced in the elderly (Grubeck-Loebenstein, B., and Wick, G., Adv Immunol 80:243-28, 2002). To determine whether the increased Aβ responses seen in our elderly and Alzheimer's disease groups were also observed for other T-cell responses, we examined T-cell responses to anti-CD3/CD28 antibodies and to a universal antigen that stimulates T cells through the T-cell receptor (TCR), glatiramer acetate (GA) (Teitelbaum, D. et al., Eur J Immunol 1:242-248, 1971). GA is a synthetic random polypeptide that stimulates a broad spectrum of HLA class II DR-restricted CD4+ T-cell populations in human PBMCs (Duda, P. W., Krieger, J. I., Schmied, M. C., Balentine, C., and Hafler, D. A., J Immunol 165:7300-7307, 2000) and T-cell reactivity to GA in humans does not require prior in vivo priming. Thus, anti-CD3/CD28 stimulation and primary T-cell responses to GA were measured as an indication of global T-cell activation and proliferation. As shown in Table 2 below, proliferative responses to anti-CD3/CD28 or GA were not increased in the elderly or patients with Alzheimer's disease.

In this study, αCD3/CD28 stimulation was measured as follows. PBMCs were cultured with 1 μg/ml soluble αCD3 plus αCD28 or cell culture medium alone, pulsed with [³H] thymidine on day 2 after stimulation and incorporation measured 12 hr later. For glatiramer acetate stimulation, cells were cultured with 50 μg/ml GA or cell culture medium alone, and pulsed with [³H] thymidine on day 6. Mean cpm=average cpm for each group. The average cpm for cells with culture medium alone was 825. PBMCs from all subjects stimulated with αCD3/CD28 showed proliferation. For GA, 90% of the subjects (from all groups tested) showed proliferation (SI³ 5). Proliferative responses were calculated from these subjects. Two-tailed p values were calculated for the mean cpm using the alternate Welch test, assuming Gaussion populations with unequal SD's.

TABLE 2
Proliferation of peripheral blood mononuclear cells
stimulated with anti-CD3/CD28 or glatiramer acetate.

| | | | Alzheimer's |
|---|---|---|---|
| | Adult | Elderly | disease subjects |
| α CD3/CD28 | 23,593 ± 5,810 | 15,855 ± 3315 | 11,377 ± 3270 |
| (Mean cpm ± SEM) | (n = 10) | (n = 15) | (n = 13) |
| P value (vs. adults) | | 0.27 | 0.09 |
| Glatiramer acetate | 35,342 ± 7,893 | 27,311 ± 4,781 | 19,403 ± 3,078 |
| (GA) | (n = 13) | (n = 30) | (n = 52) |
| (Mean cpm ± SEM) | | | |
| P value (vs. adults) | | 0.4 | 0.08 |

Indeed, there was a trend for Alzheimer's disease patients to have less T-cell reactivity to both anti-CD3/CD28 and GA, as compared to the adult healthy group (p=0.09, 0.08). This finding suggests the T-cell reactivity observed for Aβ among healthy elderly and Alzheimer's disease patients was not related to a generalized increase in T-cell responses of the subjects we tested.

T-Cell Epitopes are Located in Aβ16-30, Aβ19-33, and Aβ28-42.

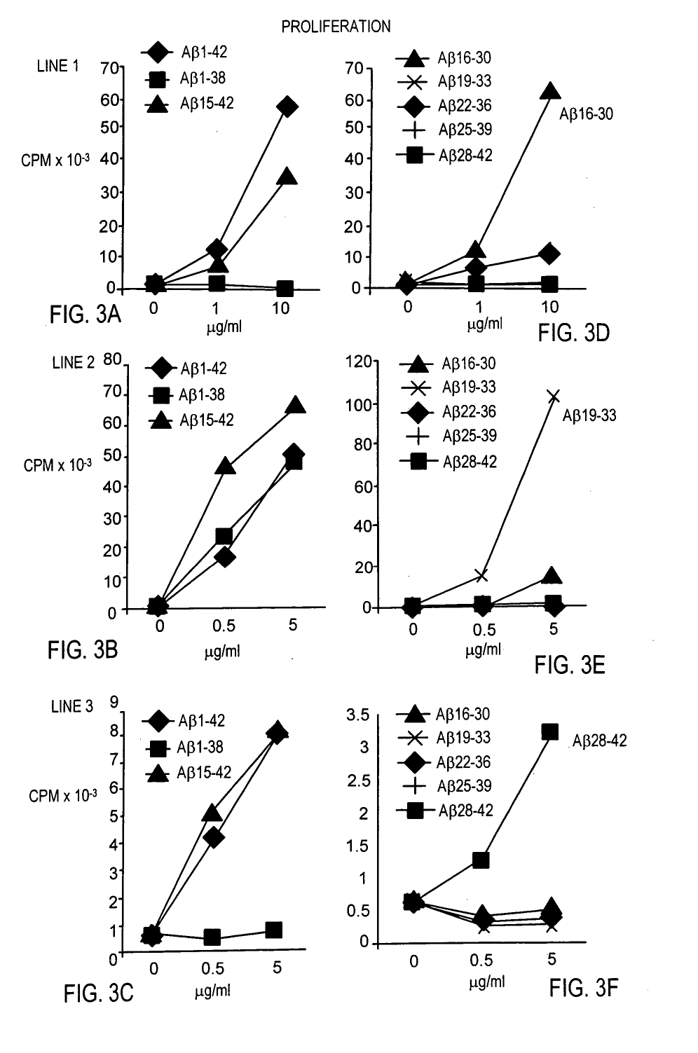

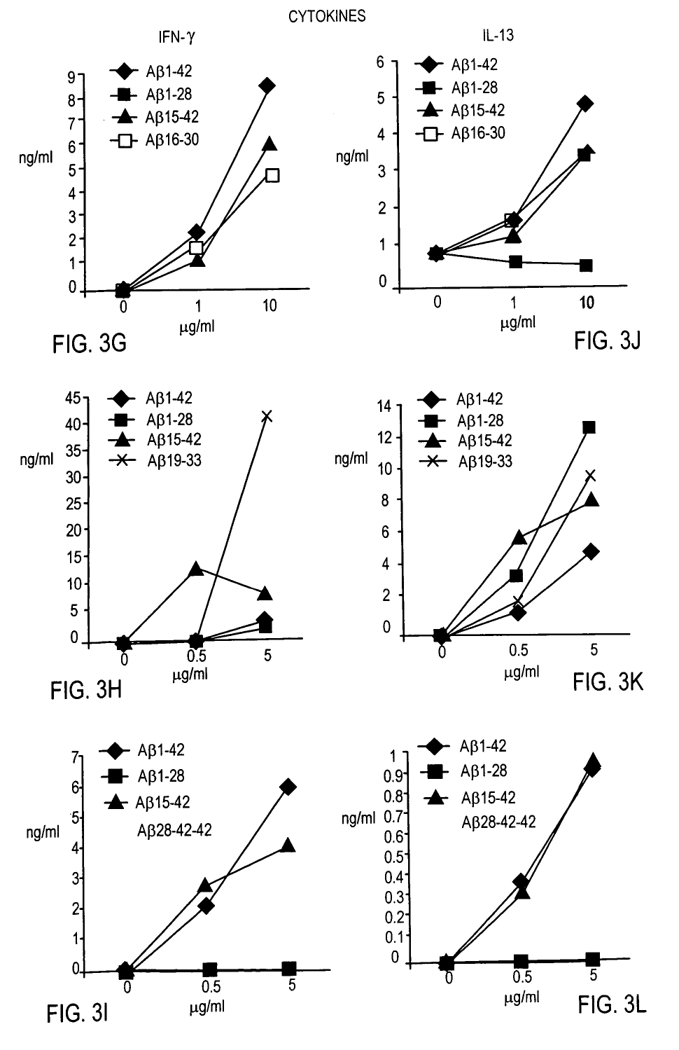

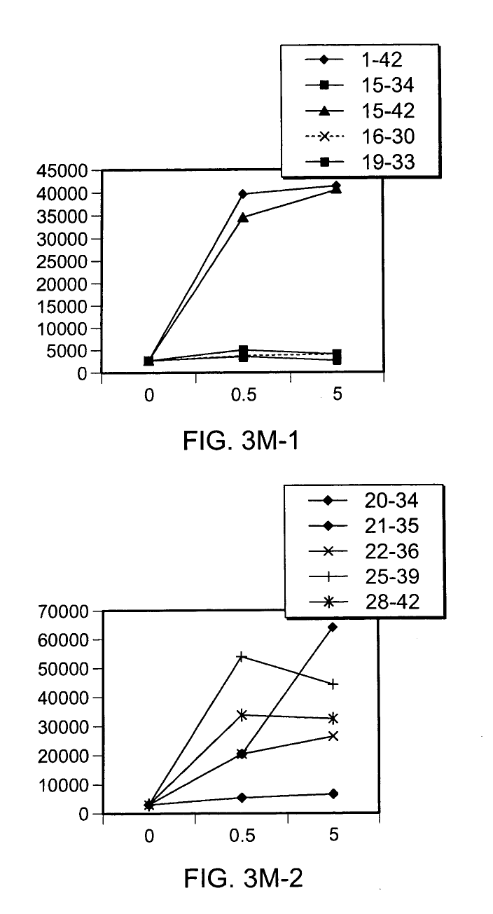

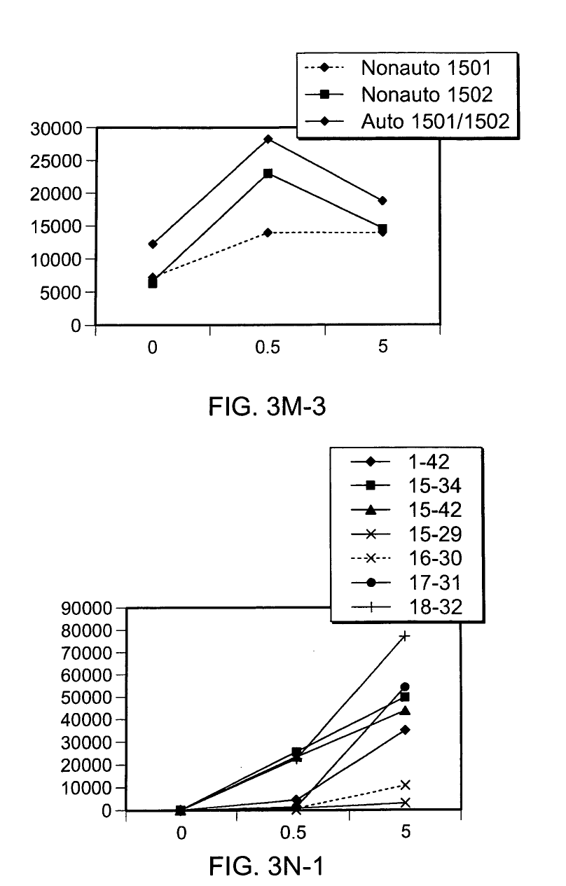

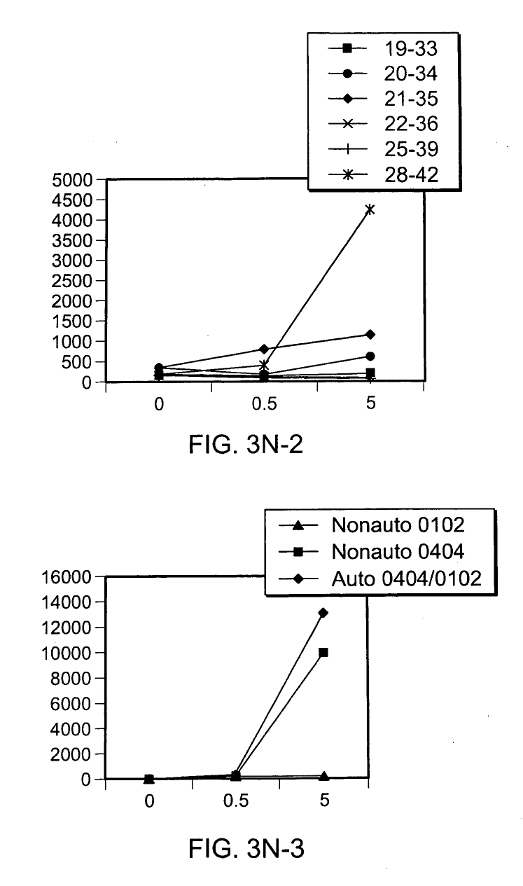

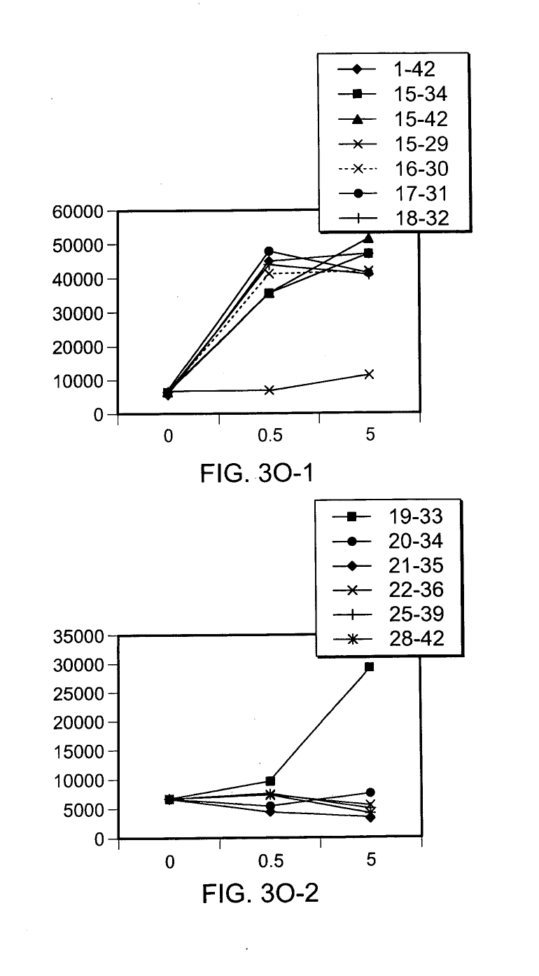

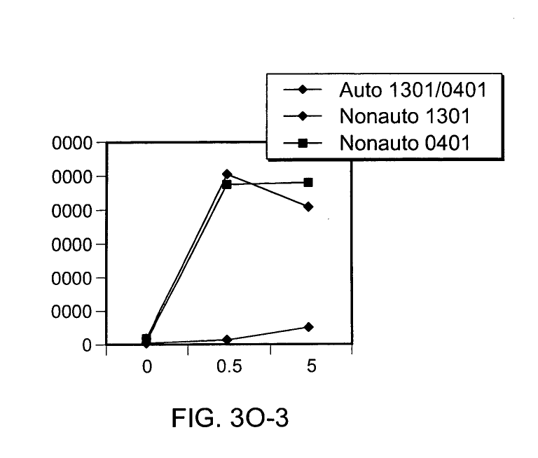

The specificity and reactivity of each T-cell line was initially determined by a dose-dependent proliferation assay using Aβ1-42 and two overlapping Aβ fragments, Aβ1-28 and Aβ15-42. Proliferation of 3 representative Aβ1-42 T-cell lines from 3 different subjects is shown in FIG. 3. T-cell epitopes were located in the C-terminus (Aβ15-42) in lines 1-3 (see FIGS. 3a-c) and also in the N-terminus (Aβ1-28) in line 2 (see FIG. 3b). Epitope specificity of these 3 T-cell lines using 5 overlapping peptides within Aβ15-42 was further investigated. CD4 T-cell epitopes were identified primarily in the Aβ15-42 peptide, which is segregated from the dominant B-cell epitopes identified in Aβ1-15.

Of the several peptides within this region that induced T-cell proliferation, the Aβ16-30 peptide was the most common epitope. As shown in FIG. 3d-f, T-cell epitopes were located in Aβ16-30, Aβ19-33, and Aβ28-42, respectively. Table 3 below summarizes Aβ epitope analyses of T-cell lines obtained from healthy subjects (6 adults and 12 older individuals) and 6 patients with Alzheimer's disease.

For these experiments, Aβ1-42-reactive T-cell lines were obtained from 24 different subjects (6 adults, 12 healthy older individuals, and 6 Alzheimer's disease patients) from the split-well assay and maintained in vitro as described in Methods. After 2-3 rounds of stimulation with Aβ1-42 and autologous PBMCs, T-cell mapping was obtained by stimulating the cells with Aβ1-28 and Aβ15-42 followed by fine mapping of 22 T-cell lines from 20 different subjects, using 15 amino acid long overlapping peptides of either Aβ1-28 or Aβ15-42.

TABLE 3
Distribution of Aβ T-cell epitopes in human PBMCs.

| | Number (percent) |
|---|---|
| Aβ1-28 | 2 (8%) |
| Aβ15-42 | 22 (92%) |
| Fine specificity | |
| Aβ7-21 | 1 (4%) |
| Aβ16-30 | 10 (42%) |
| Aβ16-33 | 6 (25%) |
| Aβ28-42 | 5 (20%) |

Aβ-reactive T-cell lines were identified that were specific to Aβ16-30, Aβ19-33 and Aβ28-42, with the highest frequency of positive T-cell lines induced by Aβ16-30. It should be noted that those T-cell lines reactive to Aβ28-42 were unreactive to Aβ1-40. HLA restriction was tested in 8 representative positive subjects bearing different HLA class II alleles. T-cell proliferation of these 8 Aβ-reactive T-cell lines was completely blocked in the presence of the monoclonal anti-HLA-DR antibody LB3.1 but not by antibodies to DP or DQ, the data of which is not shown. Other epitopes are provided for in FIGS. 3m-o.

Cytokine Profile of Aβ Reactive T-Cells Provides Expression of IL-15 and IL-13, Th2 Type Cytokines

To investigate functional properties of Aβ-reactive T cells, cytokine secretion was measured 48 hr following stimulation with Aβ1-42 or with its various overlapping fragments in the same 3 representative cell lines used for epitope mapping (see FIG. 3). T-cell lines 1, 2, and 3 secreted high amounts of IFN-γ and lower amounts of IL-13 when stimulated with Aβ1-42 or C-terminal Aβ fragments between residues 5 and 42 (see FIGS. 3g-l). In the one line that had shown reactivity to the N-terminal Aβ1-28 peptide (see FIG. 3b), Aβ1-28 also induced the secretion of high levels of IL-13, but not IFN-γ (see FIGS. 3h, k). Similar patterns of cytokine secretion were observed in 16 T-cell lines obtained from different subjects. Significant levels of IL-4, IL-10 and TGF-β, could not be detected upon stimulation with any of the Aβ epitopes tested.

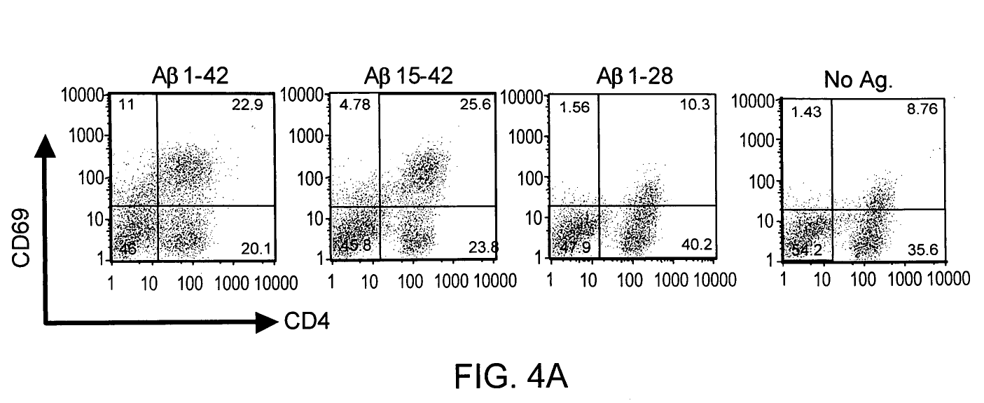

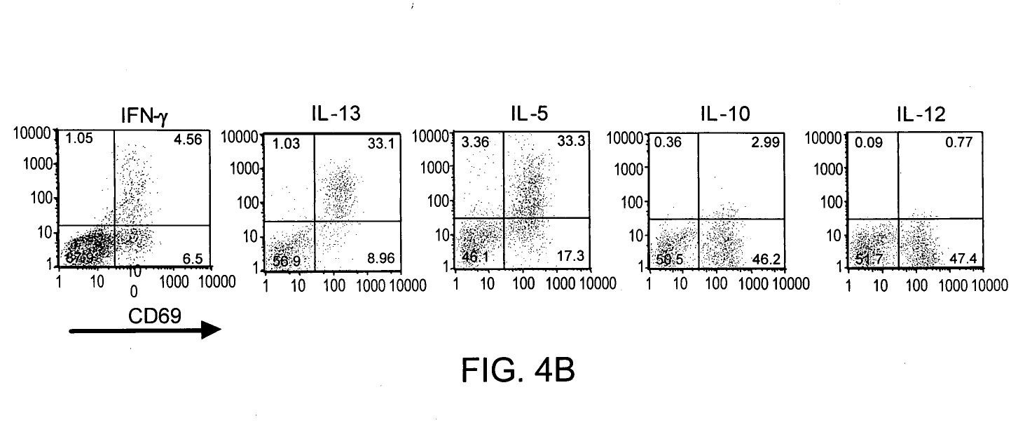

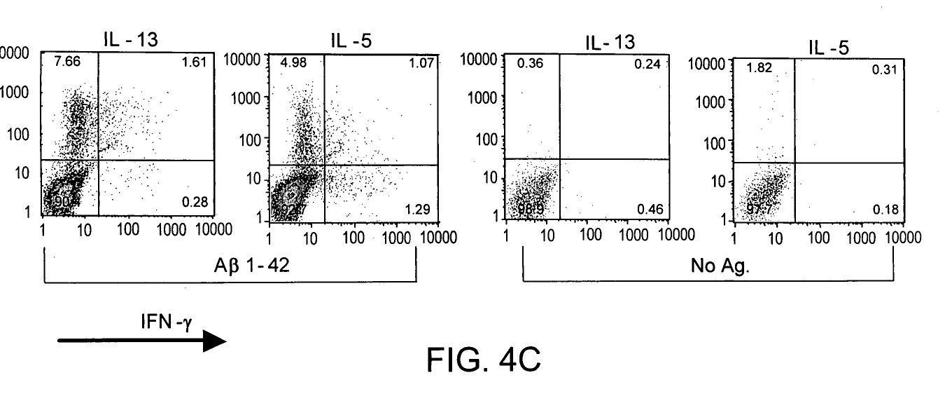

To further determine the phenotype of Aβ-reactive T cells, a representative T-cell line also was tested by FACS for Th1 and Th2 cytokine expression. Resting T cells were stimulated with irradiated autologous PBMCs in the presence of Aβ1-42, Aβ15-42, or Aβ1-28 and analyzed by FACS 30 hr later. In FIG. 4A we measured activation of CD4+ T cells for A 1-42, Aβ15-42, and Aβ1-28 and found increased expression of the CD69 activation marker on CD4+ cells stimulated with A-1-42 and 15-42, but not with Aβ1-28. We then tested the cytokine profile of the activated T cells as identified by CD69 expression (see FIG. 4B) and found that cells expressed IL-5 (33.3%), IL-13 (33.1%) with a smaller number of cells expressing IFN-γ (4.56%) and IL-10 (2.99%) and very few cells expressing IL-12 (0.77%). We further asked whether the cytokine profiles represented primarily Th2 lineages by testing for the expression of INF-γ and IL-13/IL-5 in the same cells. We found that the majority of IL-13 and IL-5 producing cells did not make IFN-γ, though we could identify a small number of double positive (IL-13/IL-5 and IFN-γ) cell populations (see FIG. 4C).

Because the T-cell epitopes were most frequently localized to Aβ16-30, we further mapped this epitope for each of its constituent amino acids by using alanine-substituted peptides in the T-cell proliferation assay (the naturally occurring alanines at positions 21 and 30 were changed to glycines). Substitution of phenylalanine-20, glutamic acid-22, and aspartic acid-23 with alanines blocked or significantly decreased T-cell proliferation at all 3 concentrations used, whereas substitutions of phenylalanine-19, valine-24 and glycine-29, respectively, had inhibitory effects only at 0.1 or 1 μg/ml (Table 4). Substitution of lysine-16, alanine-21, serine-26 or aspargine-27 increased T-cell proliferation by more than 2-fold (Table 4).

In this experiment, T cells specific to Aβ16-30 were stimulated in the presence of alanine/glycine substituted peptides and autologous PBMCs. T-cell proliferation was measured by [³H]-thymidine incorporation 72 hr after stimulation. Numbers represent average cpm of each antigen concentration tested (average cpm in the absence of antigen was 911).

TABLE 4
Proliferation of Aβ16-30-specific T cells with
alanine/glycine-substituted peptides

| | | | | Effect on T-cell |
|---|---|---|---|---|
| Peptide (μg/ml) | 0.1 | 1 | 10 | proliferation |
| Aβ16-30 | 1648 | 6780 | 17339 | |
| Substituted | | | | |
| Phe 20 Ala | 1188 | 2239 | 4216 | ↓↓↓ |
| Glu 22 Ala | 1145 | 840 | 517 | |
| Asp 23 Ala | 1145 | 1685 | 612 | |
| Phe 19 Ala | 986 | 4354 | 17943 | ↓ |
| Val 24 Ala | 944 | 6160 | 19869 | |
| Gly 29 Ala | 622 | 3014 | 14513 | |
| Leu 17 Ala | 2242 | 11805 | 24480 | ↑ |
| Val 18 Ala | 7216 | 17427 | 26814 | |
| Gly 25 Ala | 2426 | 13763 | 24979 | |
| Lys 28 Ala | 5050 | 15475 | 29156 | |
| Ala 30 Gly | 2123 | 12027 | 15395 | |
| Lys 16 Ala | 5357 | 22304 | 36456 | ↑↑ |
| Ala 21 Gly | 12901 | 29634 | 36448 | |
| Ser 26 Ala | 18808 | 36287 | 38335 | |
| Asn 27 Ala | 23636 | 36588 | 34983 | |

Analysis of Aβ Epitopes in HLA-DR Restriction in Human Subjects:

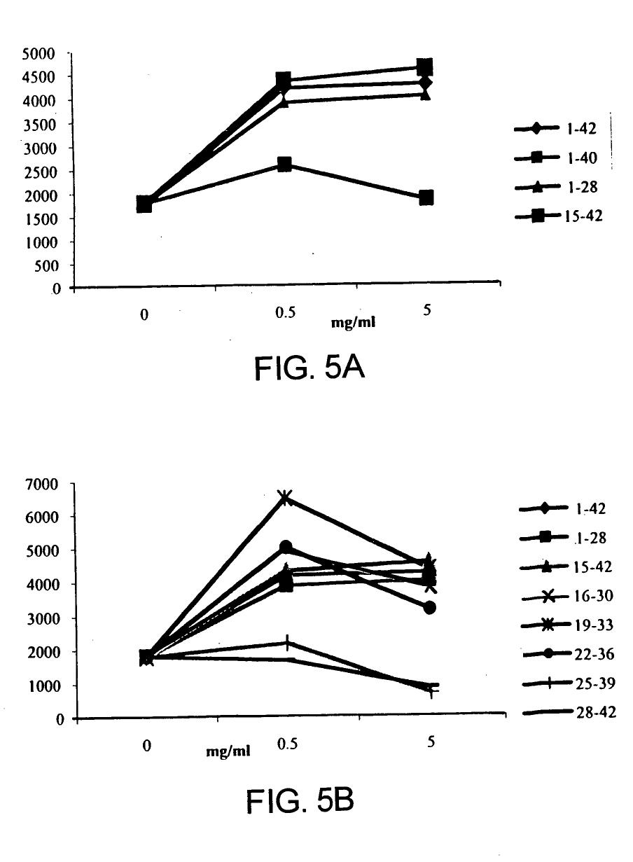

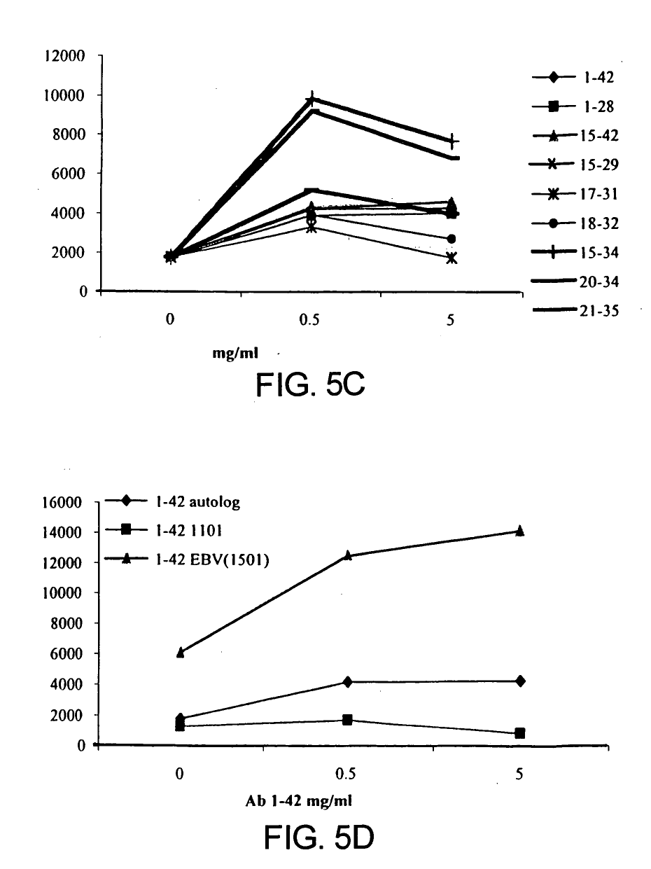

The specificity and reactivity of each T-cell line was initially determined by a dose-dependent proliferation assay using Aβ1-42 and two overlapping Aβ fragments, A1-28 and Aβ15-42. Proliferation of a representative A11-42 T-cell line is shown in FIG. 5A where the T-cell epitope was located in both C-terminus and N-terminus (Aβ1-28/15-42). We then further investigated epitope specificity of the T-cell line using 5 overlapping peptides within Aβ15-42. As shown in FIG. 5B, T-cell epitopes were mainly located in Aβ19-33. Further analysis of the main T-cell epitope between 20-34 is shown in FIG. 5C.

To test HLA class II restriction, T-cell proliferation was examined using autologous EBV line bearing HLA DR1 1501 and 1101 alleles. As shown in FIG. 5D, the 1501-specific EBVs induced proliferation of A-beta reactive T cells, demonstrating that 1501 HLA allele presents these Aβ epitopes to T cells in this subject.

Aβ Immunogenicity is Determined by Epitope Specificity.

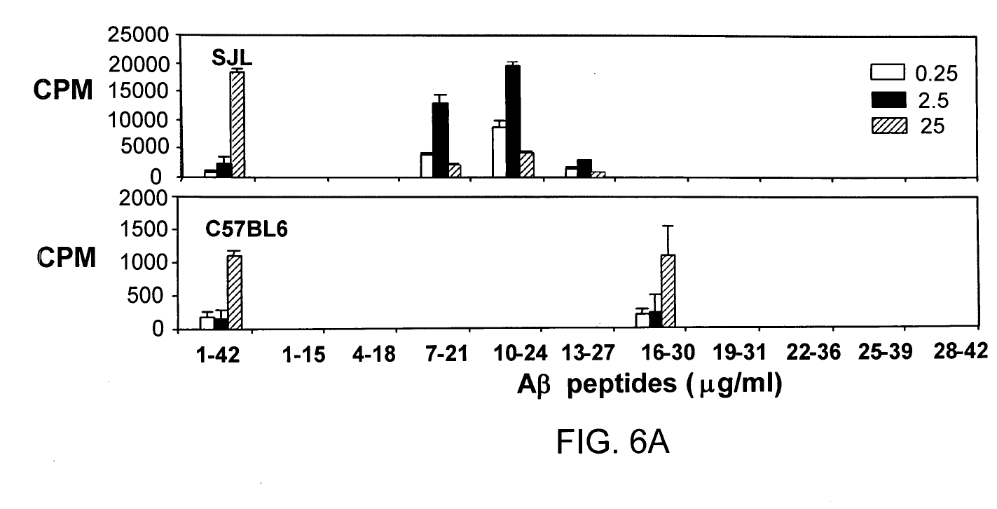

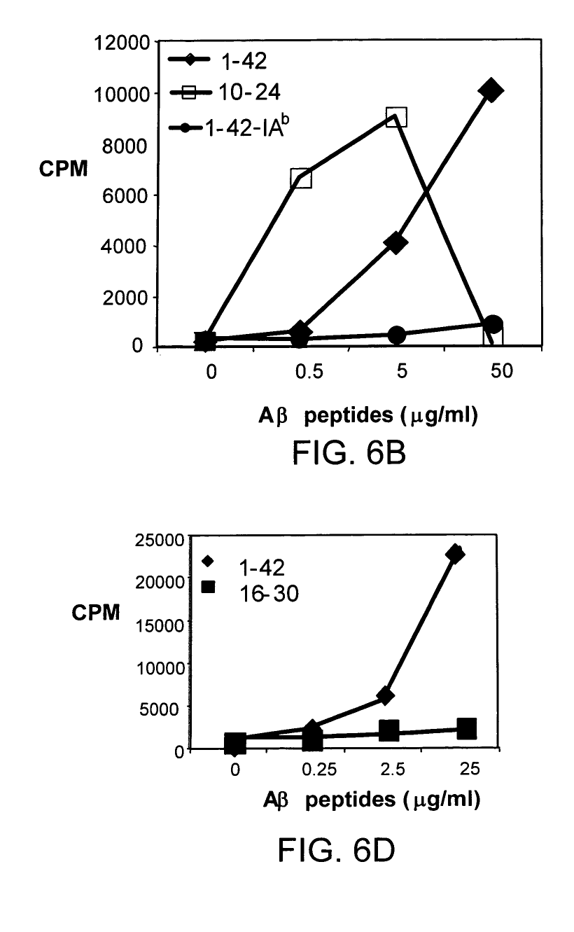

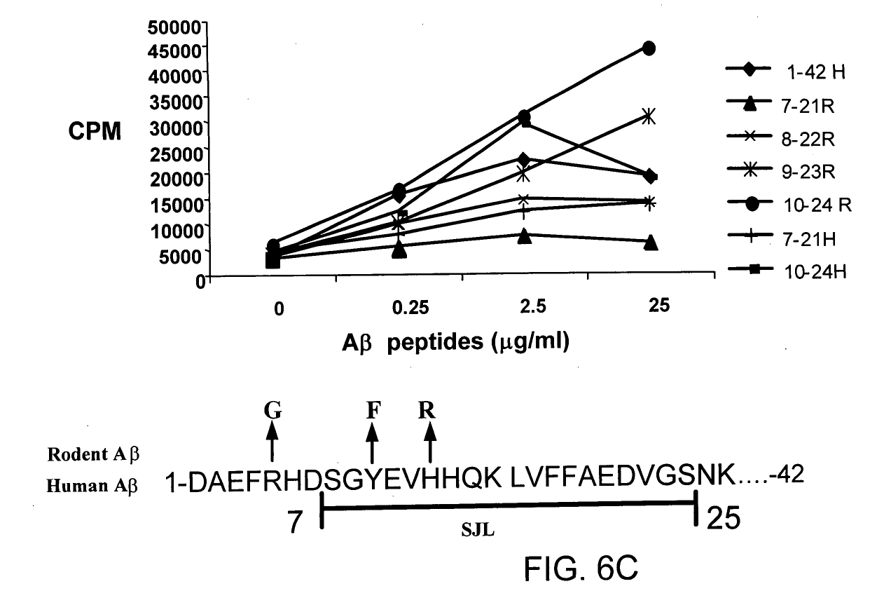

To analyze genetic control of Aβ immunogenicity, we immunized C57BL6 and SJL mice (H2^{b} and H2^{s} MHC class II haplotypes, respectively) with human Aβ1-42 in CFA and assessed proliferative responses in popliteal draining lymph nodes (PLN). Aβ-specific T-cell proliferation was significantly higher in LN from SJL than from C57BL6 mice (FIG. 1A). To determine the specific T-cell epitopes in each of the strains, T-cell proliferation also was measured using 10 overlapping peptides of Aβ1-42 (see Methods). Peptides 7-21, 10-24, and 13-27 induced T-cell proliferation in the SJL-derived lymphocyte cultures (FIG. 6A), whereas only peptide 16-30 induced proliferation in C57BL6 mice (FIG. 6A). The highest proliferative response was obtained using 2.5 and 25 μg/ml peptide in SJL and C57BL6 mice, respectively (FIG. 6A). Responses to peptides 10-24 in SJL and 16-30 in C57BL6 mice were equivalent to those induced by Aβ1-42 (FIG. 6A).

Low T-cell reactivity in C57BL6 could result from low-affinity T-cell epitope presented by the specific MHC class II allele. To determine if the IA^{b} class II allele presented by C57BL6 mice indeed load a low affinity T-cell epitope, we have used the highly immunogenic NOD mice and compared Ab-specific T-cell responses to NOD congenic mice expressing the IA^{b} class II allele. NOD and NOD congenic mice were immunized with Aβ1-42 and T-cell proliferation was measured. NOD mice had a high Aβ-specific T-cell proliferation response to Aβ1-42, for which the T-cell epitope was between Aβ residues 10 and 24 (FIG. 6B). Only slight T-cell proliferation was induced by Aβ7-21 and to a lesser extent by Aβ13-27, but not by any other Aβ peptide (not shown). By contrast, immunization of NOD-IA^{b} congenic mice with whole Aβ resulted in a low T-cell proliferation response, similar to that of C57BL6 mice, suggesting that the low immunogenicity of Aβ1-42 in C57BL6 is primarily due to the T-cell epitope presented by the IA^{b} allele.

Because 3 residues are different in rodent Aβ, we sought to determine whether the high T-cell responses obtained in SJL and NOD mice immunized with human Aβ1-42 were also specific to the self Aβ peptide. Thus, SJL mice were immunized with rodent Aβ1-42 and T-cell responses to rodent and human Aβ peptides were tested in vitro. Similar T-cell proliferation was obtained when human or rodent Aβ7-21, and human or rodent Aβ10-24 were used as the stimulating peptide (FIG. 6C). In addition, there was gradual increase in T-cell proliferation using Aβ7-21, Aβ8-22, Aβ9-23, and Aβ10-24 peptides as antigens (FIG. 6C), suggesting that the full-length Aβ epitope in SJL mice is located between residues 10 and 24 and that immunization with either human or rodent Aβ1-42 evokes T-cell responses to this peptide. Recall T-cell responses to human Aβ1-42 were then measured in vitro following immunization of SJL mice with human Aβ10-24. As shown in FIG. 6D, LN-derived T cells from SJL mice immunized with Aβ10-24 proliferated in vitro when stimulated with human Ab1-42 but not Ab16-30. Overall, these data demonstrate that different Aβ-specific CD4 T-cell epitopes presented by different MHC class II alleles have a significant impact on Aβ immunogenicity.

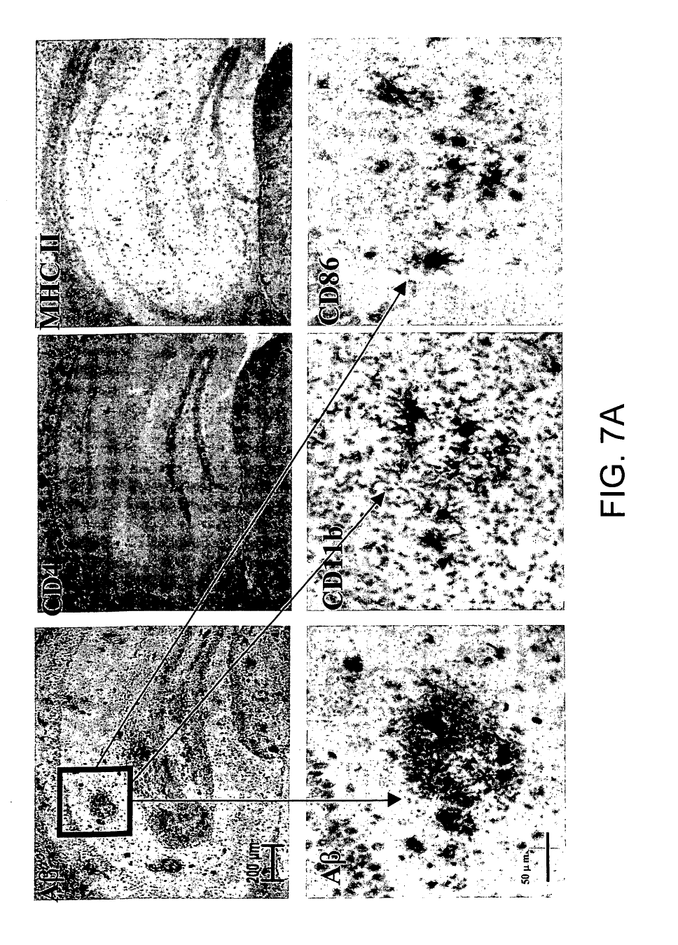

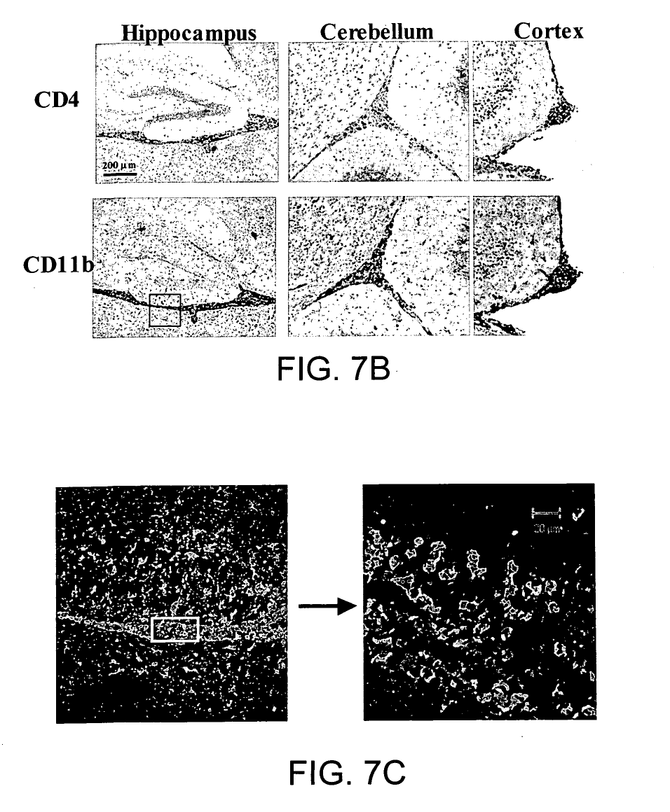

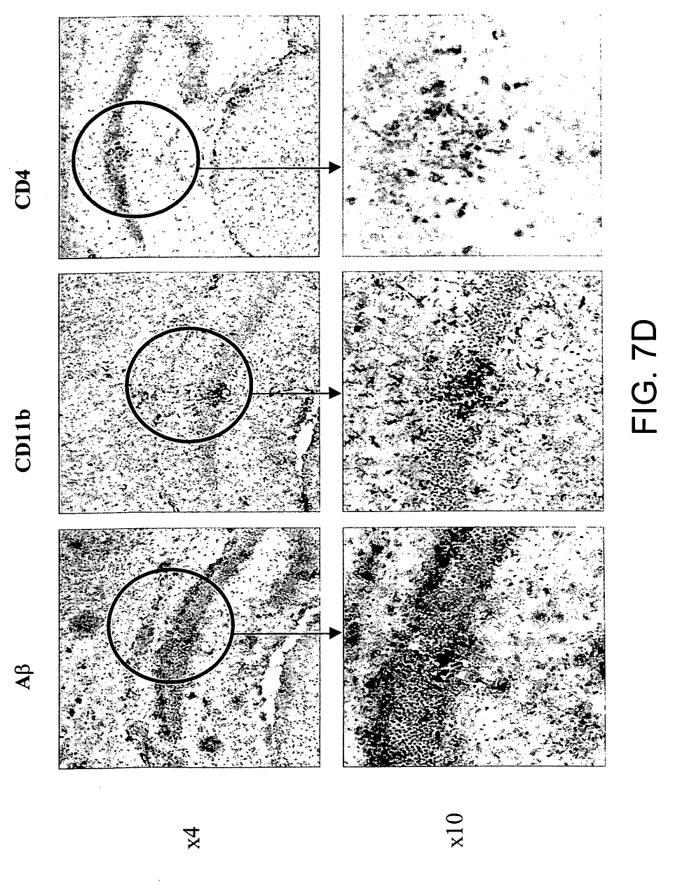

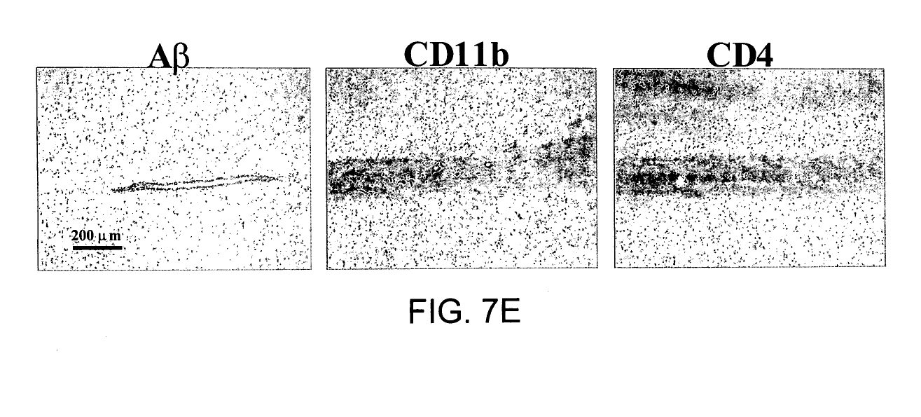

Immunization with the T-cell epitope Aβ10-24 results in transient encephalitis in APP/IFN-g double Tg mice. As shown in FIG. 6, I-A^{s} but not I-A^{b} MHC class II haplotype was essential to mount a significant Aβ-specific T-cell response. To determine whether carrying the IA^{s} allele is sufficient to induce T-cell activation and migration to the CNS of APP-tg mice, B6SJLF1 APP-tg mice line J20, see Methods) were immunized with Aβ10-24 in CFA followed by intravenous injection of PT at the time of immunization and 48 hr later. Although Aβ-specific T-cell responses were as high in APP-tg B6SJLF1 as in immunized SJL mice (data not shown), infiltrates of CD4 or CD11b cells were not observed in meningeal tissues or anywhere else in the brain (FIG. 7, Table 5).

TABLE 5
The kinetics of CD11b and CD4 cell infiltrates in the hippocampus
meningeal tissue of APP/IFN-g Tg mice compared with single-Tg mice
after immunization with Ab10-24

| | Days | | | CD4 | CD11b |
|---|---|---|---|---|---|
| Transgenic | after | | Immunization | (cells/mm² ± | (cells/mm² ± |
| Gene/s | imm. | Background | peptide | SEM) | SEM) |
| App | 12 | B6-SJL-F1 | AB₁₀₋₂₄ | 0 | 0 |
| App/INF-g | 12 | B6-SJL-F1 | AB₁₀₋₂₄ | 689 | 1111 |
| App/INF-g | 20 | B6-SJL-F1 | AB₁₀₋₂₄ | 400 | 1022 |
| App/INF-g | 30 | B6-SJL-F1 | AB₁₀₋₂₄ | 400 | 289 |
| App/INF-g | 60 | B6-SJL-F1 | AB₁₀₋₂₄ | 0 | 0 |
| App/INF-g | 12 | B6-SJL-F1 | BSA | 0 | 0 |
| INF-g | 12 | B6-SJL-F1 | AB₁₀₋₂₄ | 13 | 148 |
| App | 12 | C57BL6 | AB₁₆₋₃₀ | 0 | 0 |

Immunostaining of brain sections from immunized APP-Tg mice showed activated microglia primarily at sites of compact Aβ plaques colocalized with expression of the T-cell costimulatory molecule CD86 (FIG. 7), which is similar to the staining pattern of unimmunized APP-Tg mice (not shown).

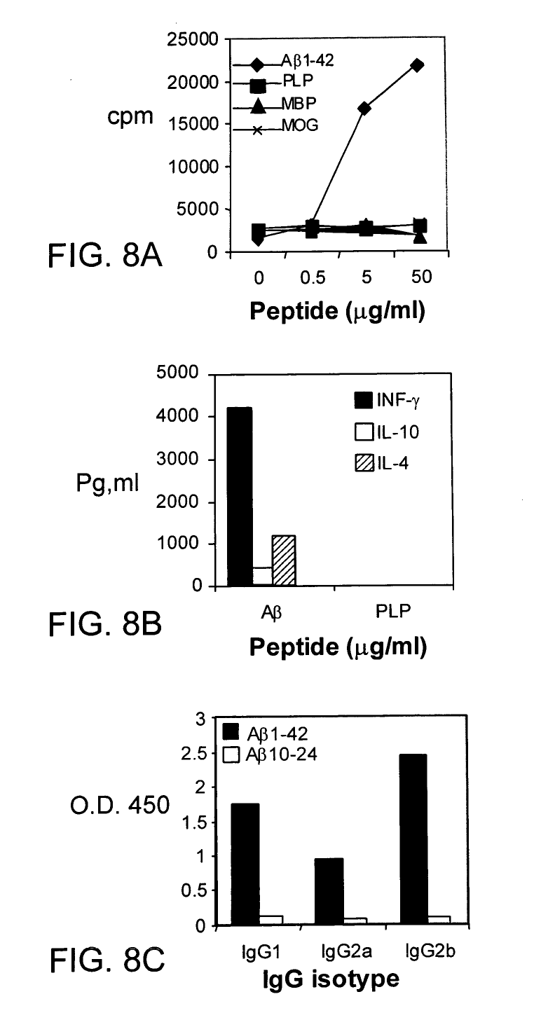

In order to determine whether a higher state of microglia activation, in addition to MCH genetic background contributed to meningoencephalitis in some patients with AD, (Mattiace, L. A., P. Davies, and D. W. Dickson. 1990. Detection of HLA-DR on microglia in the human brain is a function of both clinical and technical factors. Am J Pathol 136:1101.), we tested whether limited expression of IFN-γ in the CNS would render Aβ-specific T cells as an encephalitogenic ones. APP-Tg mice were crossed with SJL-backcrossed mice expressing IFN-g in the CNS under an MBP promoter (Renno, T., V. Taupin, L. Bourbonniere, G. Verge, E. Tran, R. De Simone, M. Krakowski, M. Rodriguez, A. Peterson, and T. Owens. 1998. Interferon-gamma in progression to chronic demyelination and neurological deficit following acute EAE. Mol Cell Neurosci 12:376.). Immunization of APP/IFN-γ double tg mice with Aβ10-24 resulted in a strong meningoencephalitis as early as 12 days after immunization, shown by immunolabeled CD11b+ macrophages and CD4+ T cells, primarily in the hippocampus, but also in the cortex and the cerebellum (FIG. 8A). A three-color staining of sections from this time point after immunization showed Aβ deposits (blue) in the hippocampus region and accumulating macrophages (green) and T cells (red) in the adjacent meningeal tissues (FIG. 8B). Activated microglia and macrophages (presumably migrating from the meninges) were colocalized with accumulated Aβ plaques (FIG. 8B).

APP/IFN-γ tg mice that were immunized with Aβb 10-24 were analyzed by immunohistochemistry 20 and 30 days after immunization for immune infiltrates associated with Aβ plaques. In contrast to day 12 post-immunization, when CD4 T cells and CD11b macrophages were located primarily in meningeal tissues, on day 20 CD4 and CD11b cells were located at sites of Aβ plaques in the hippocampus, and fewer were observed in the meninges (FIG. 8C, see arrows). Apparently, these CD4 T cells migrated mainly to compact Aβ plaques (FIG. 8C, see arrows), sites that were occupied by associated with Aβ plaques. In contrast to day 12 post-immunization, when CD4 T cells and CD11b macrophages were located primarily in meningeal tissues, on day 20 CD4 and CD11b cells were located at sites of Aβ plaques in the hippocampus, and fewer were observed in the meninges (FIG. 8C, see arrows). Apparently, these CD4 T cells migrated mainly to compact Aβ plaques (FIG. 8C, see arrows), sites that were occupied by activated microglia prior to immunization. On day 30, reduced numbers of CD4 and CD11b infiltrates were detected in meningeal tissues of the brain as well as at sites of Aβ plaques (Table 5). Immune infiltrates were not observed in the spinal cord of these mice at any time (FIG. 8D). As shown in FIG. 12 (Table 1), CD4 and CD11b cell infiltrates were observed in brain sections of APP/IFN-γ double tg mice immunized with Aβ but not in APP/IFN-γ Tg mice immunized with BSA or APP single-Tg mice having the same genetic background. In 1 out of 3 IFN-γ single-Tg mice, a few infiltrating cells were observed in the meninges (Table 5). In contrast to APP/IFN-γ Tg mice, these cells did not migrate from the meninges to the parenchymal tissue (not shown).

Ab10-24-Induced Encephalitis is Mediated by Specific Th1 Cells.

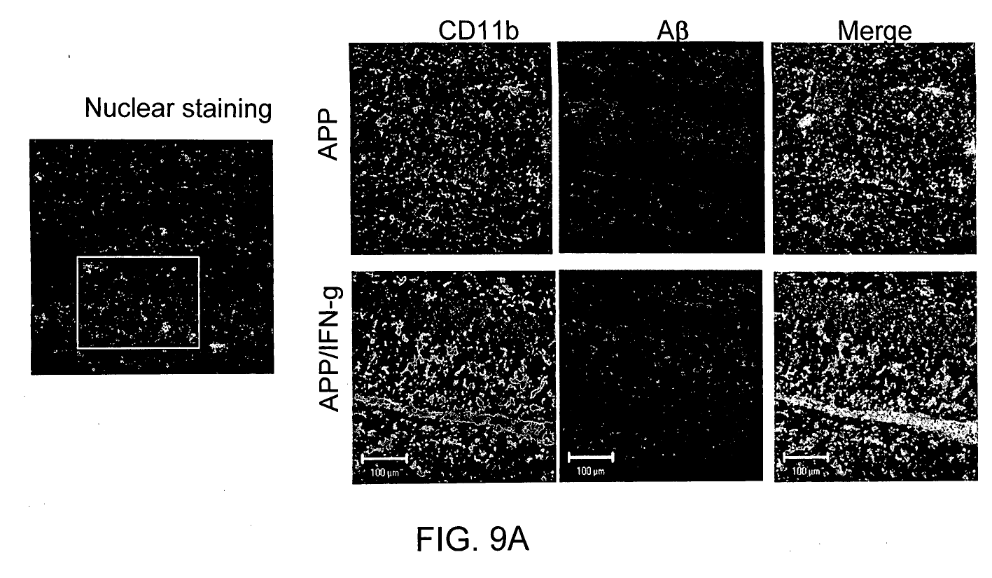

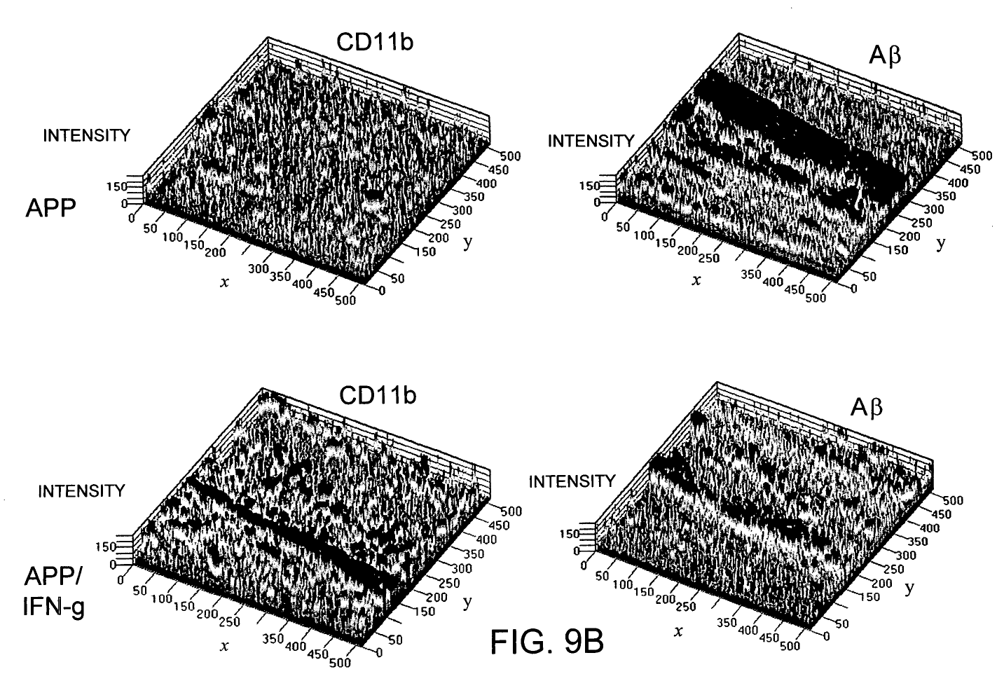

Peripheral immune responses to Aβ also were characterized in vitro on days 14, 20, and 30 after Aβ immunization. Proliferation of spleen-derived T cells was induced by Aβ but not by myelin peptides such as PLP, MOG, and MBP, all known to induce EAE in these mice strains (FIG. 9A). High amounts of IFN-γ and low amounts of IL-4 and IL-10 were secreted by these Aβ-specific T cells, indicating that primarily a Th1 but not Th2 type of immune response was elicited (FIG. 9B). Serum isolated from these Aβ10-24 immunized APP-tg mice had no Aβ binding antibodies, in contrast to Aβ10-42 immunized mice, in whose serum high titers of all 3 isotypes IgG1, IgG2a, and IgG2b were detected (FIG. 9C). Taken together, these data suggest that upon immunization with the T-cell epitope Aβ10-24, Aβ-reactive T cells migrate specifically to brain regions where Aβ is accumulated and trigger a proinflammatory response that lasts for about 30 days. As Aβ10-24 lacks the dominant B-cell epitopes site, the immune response did not include production of Aβ antibodies after a single immunization.

Clearance of Aβ in the Hippocampus is Enhanced by Activated Microglia/Macrophages.

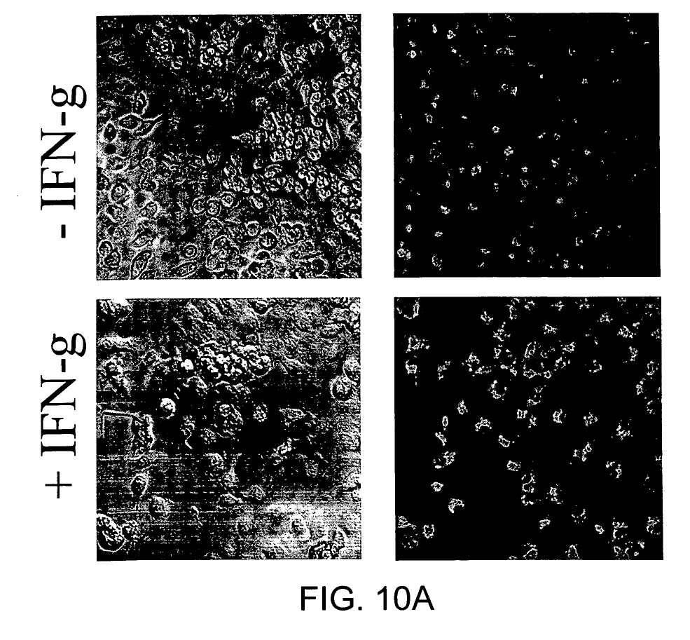

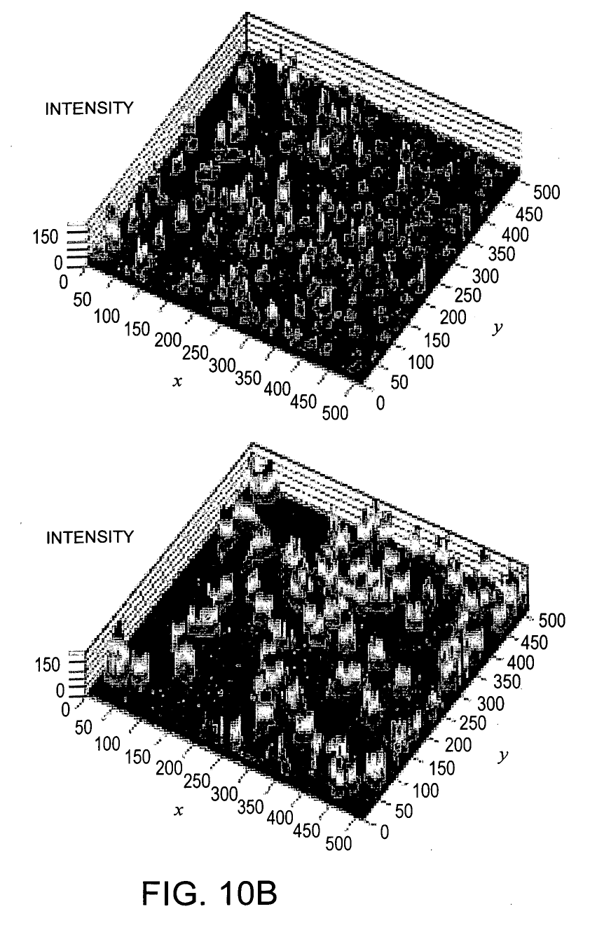

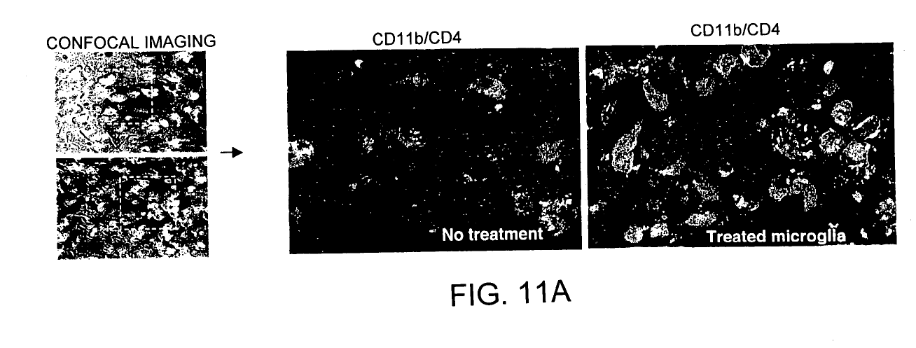

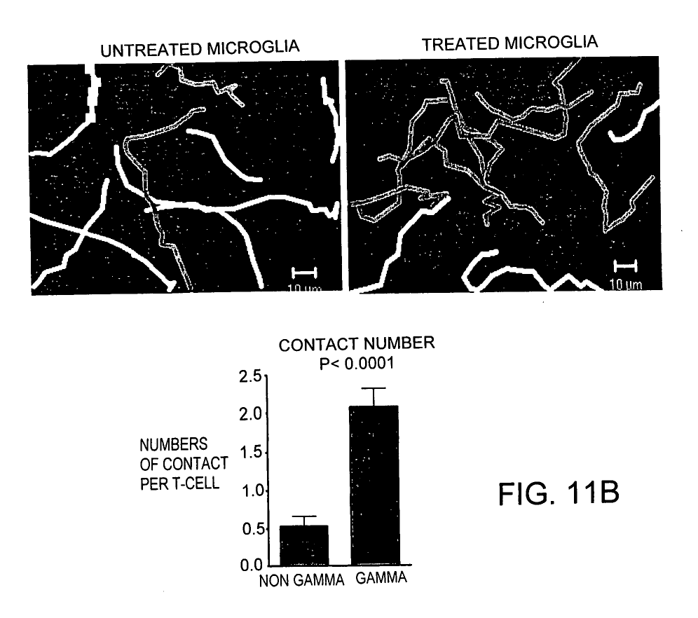

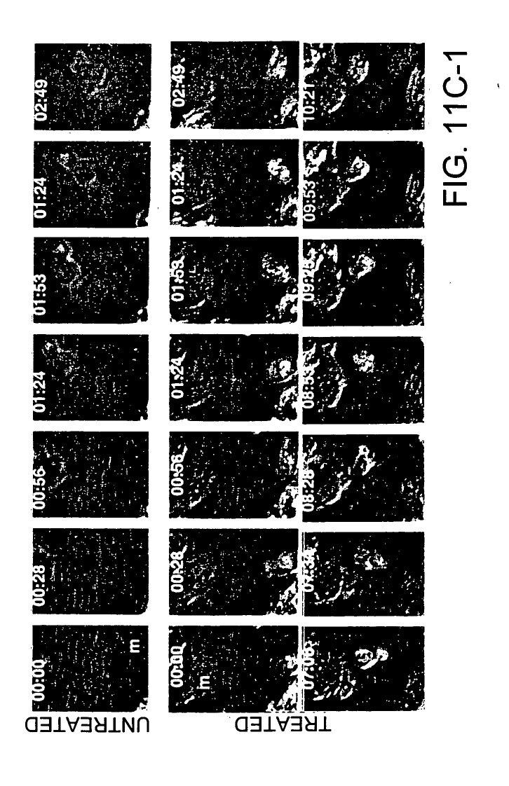

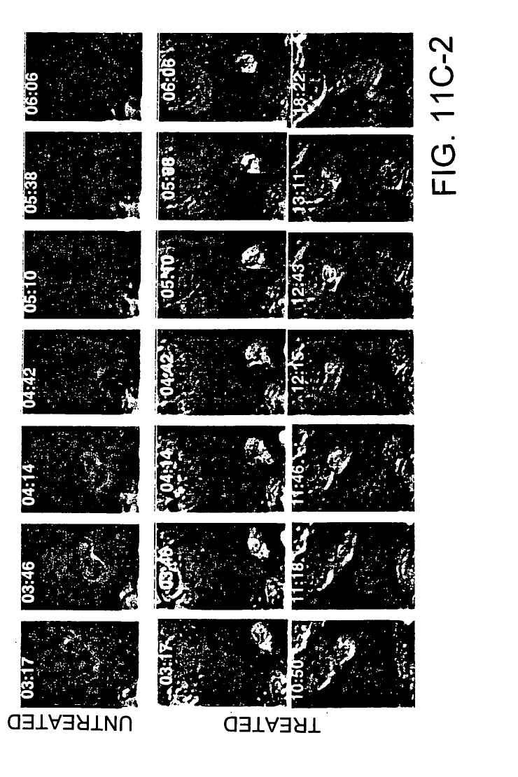

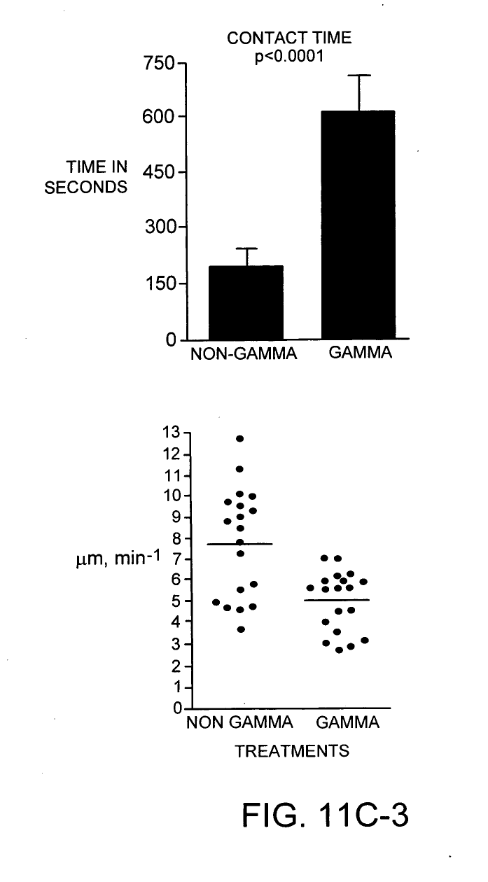

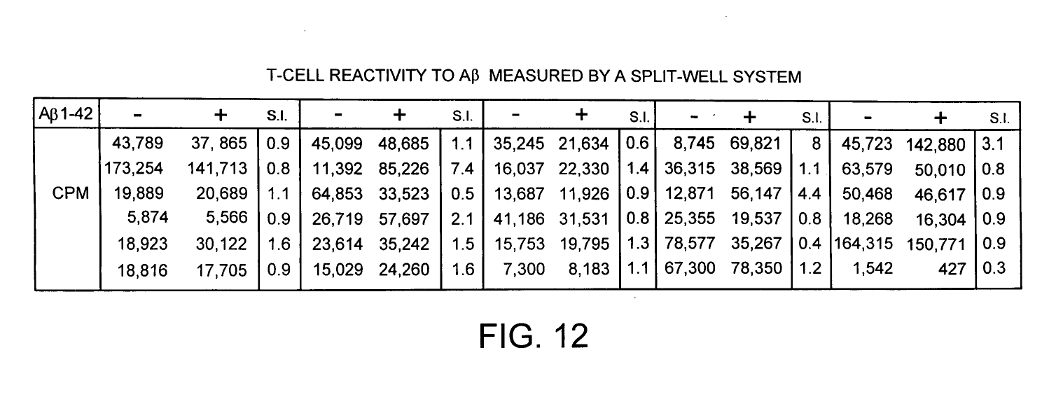

As shown in FIGS. 8 and 9, a single immunization with Aβ10-24 resulted in a panel a). Brain sections of APP-Tg mice showed a few activated microglia primarily at sites of neuritic plaques but not diffuse plaques (FIG. 10A, panels b-d). In contrast, submeningeal tissues in the hippocampus of APP/IFN-γ mice immunized with Aβ10-24 had many highly activated microglia/macrophages that colocalized with Aβ deposits. These deposits contained significantly decreased amounts of Aβ (FIG. 10A, panels e-g). A three-dimensional intensity analysis representing the fluorescence of Aβ and CD11b staining in panels b,c,e,f shows clear reduction in the intensity of Aβ staining in contrast to increased intensity of the CD11b staining (FIG. 10B). The mean intensity of Aβ staining in these hippocampus submeningeal areas between days 12 and 20 after a single immunization with Aβ-24 was 36.8±6.3 in APP/IFN-γ mice compared with 76.2±16.4 in APP non-immunized mice at similar age (n=5/group, P=0.05).

IFN-γ Enhances Microglia Motility and Uptake of Ab, and Microglia-Induced T-Cell Activation.

The meningoencephailis we observed was completely dependent on expression of IFN-g in the CNS. High amounts of IFN-γ produced by T cells following Aβ immunization did not induce encephalitis in APP-Tg mice (FIG. 7A). We therefore established an in vitro system to examine early events associated with microglia activation by IFN-γ and their contribution to uptake of Aβ and specific T-cell activation. Primary cultures of microglia were prepared and live images were obtained by a confocal microscope, as described in Methods. FITC-labeled Aβ was added to the culture for 30 min, cultures were carefully washed, and live images were taken for 30 minutes. Aβ uptake (green) was significantly enhanced in microglia pretreated with IFN-g as compared to untreated cultures as indicated by the fluorescence images (sum of 30 min live imaging) and the related intensity analysis (FIG. 10A). Furthermore, filipodia formation as an indication of microglia motility were longer and occurred with higher frequency in the IFN-γ treated cultures. (FIG. 10B).

We then co-cultured microglia and resting Aβ-reactive T cells and examined the effect of IFN-γ on T-cell motility and synapse formation in the presence of Aβ1-42 as an antigen. Microglial cells were sorted and cultured for 12 hours in the presence and absence of IFN-γ. After washing the cultures from residual IFN-γ, Aβ1-42 and resting Aβ-reactive T cells were added to the cultures for 6 hours and live images were taken for 30 min. Although the same number of Aβ-reactive T cells were added to the microglial (red) cultures, higher density of T cells (green) were observed in IFN-γ-treated than in untreated microglia (FIG. 11A). Representative areas were then extracted (shown in higher power) and analyzed for the pattern of T-cell migration. In the IFN-γ-treated culture, T cells were attracted to microglial cells, indicated by the sharp turns between the cells (FIG. 11B), and made a significant higher average number of contacts with the microglial cells, as compared with the untreated microglial culture (about 2.1 versus about 0.5, P<0.001) (FIG. 11B). The motility pattern of T cells and synapse formation in IFN-γ treated and untreated microglial culture is shown in FIG. 6C and supplement 1. In contrast to the untreated culture, there was a significant increase in the motility of the T cells in the IFN-γ-treated culture which migrated to microglial cells and established contact (FIG. 11C); the contact time between the T-cell and microglial cell was 8 min 29 seconds. These contacts are short time synapse formations characteristics of an early event during T-cell stimulation.

### Materials and Methods

Antigens used in Humans: Aβ1-40 and Aβ1-42 synthetic peptides from Biosource International (Camarillo, Calif.) were examined for fibril formation in a phosphate buffer solution (pH 7.4), using quasielastic light-scattering spectroscopy (QLS). Fibril formation was measured in 3 equivalent samples of 10 μg/ml Aβ for 5 consecutive days. Slight aggregation was detected in only one sample after a 1-day incubation without significant change up to day 5. Nested Aβ peptides (Aβ1-15, Aβ4-18, Aβ7-21, Aβ10-24, Aβ13-27, Aβ16-30, Aβ19-33, Aβ22-36, Aβ25-39, Aβ28-42) and alanine/glycine-substituted peptides were synthesized at Biosource International (Hopkinton, Mass.). Aβ peptides used for T-cell assays were dissolved in DMSO (2 mg/ml). Glatiramer acetate (GA) was obtained at the Brigham and Women's Hospital pharmacy.

MHC Class II molecules: MHC Class II molecules can be developed to specific peptide and allele specifications, and can be purchased and developed commercially from Beckman Coulter Immunonics, San Diego, Calif. Additional approaches for the generation of alternative multimers of peptide/MHC complexes are described in a review article by Hugues et al. (Jornal of Immunological Methods 268:83-92 (2002)). Peptides can be attached to the MHC molecule in a variety of ways, for example through a covalent bond, or a non-covalent bond.

Human Subjects: Subjects with Alzheimer's disease (ages 65-90) were recruited from the Memory Disorders Unit at Brigham and Women's Hospital under an IRB-approved human studies protocol. The ages and educational background of the Alzheimer's disease patients were equivalent to those of the healthy older subjects, who were often the spouses of the patients. The diagnosis of Alzheimer's disease was based on NINDS-ADRDA criteria and included use of the Mini Mental State Exam (MMSE). Patients with mild to moderate Alzheimer's disease having MMSE scores between 10 and 24 were selected for this study. Patients with severe Alzheimer's disease (MMSE<10) or whose history included a severe head injury, alcoholism, major psychiatric illness, epilepsy, or learning disability were excluded from the study. Overall, 27 adults, 35 older healthy individuals, and 42 patients with Alzheimer's disease were evaluated in the study (total=104).

Split well culture system for Aβ reactivity: PBMCs were isolated from freshly drawn heparinized whole blood by Ficoll-Paque (Amersham Pharmacia Biotech, Uppsala, Sweden) gradient centrifugation, according to the manufacturer's protocol. Cells were cultured in 30 wells of 96-well round bottom plates at 2×105 cells/well in RPMI medium (containing 2.5% non-autologous serum, 4 mM L-glutamine, 10 mM HEPES, 1 mM sodium pyruvate, 10 mM nonessential amino acids) in the presence of pure human Aβ peptide. At day 5, 10 μg/ml of recombinant human IL-2 was added by replacing half of the medium with IL-2-containing medium. On day 7, medium was replaced with IL-2 free medium. On day 10, half the cells from each well were restimulated with irradiated autologous PBMCs in the presence and the absence of 10 μg/ml Aβ for 48 hr and then pulsed with (Renno, T., et al., Mol Cell Neurosci 12:376-389 (1998)) thymidine for 12 hr. Cells were then harvested and radioactivity measured (cpm). The stimulation index (SI) (cpm in the presence of Aβ divided by cpm in the absence of Aβ was determined. Positive wells are those with a stimulation index of ≧2.5 and a Δcpm≧2,000.

Primary stimulation with αCD3/CD28 or GA: For βCD3/CD28 stimulation frozen PBMCs (2×10⁵ cells/well) were cultured in 5 wells (U-bottom 96-well plate) in the presence of 1 μg/ml soluble αCD3 plus αCD28 and in additional 5 wells with cell culture medium alone, pulsed with (Renno, T. et. al., Mol Cell Neurosci 12:376-389, 1998) thymidine on day 2 after stimulation and incorporation measured 12 hr later. For glatiramer acetate stimulation, fresh PBMCs were cultured in 5 wells with 50 μg/ml GA or cell culture medium alone, pulsed with [³H] thymidine on day 6, and incorporation measured 12 hr later.

T-cell lines, fine specificity, cytokine production, proliferation, and flow cytometry: Positive wells obtained in the split-well assay were restimulated 14 days after their primary stimulation in the presence of autologous PBMCs (2×10⁵ cells/well) and Aβ1-42. T-cell lines were then supplemented with 10 μg/ml IL-2 after 48 hours and every 2 days thereafter until they were completely resting. T-cell proliferation was measured 72 hours after stimulation following a 12 hr incubation with [³H]-thymidine. Supernatants were collected 48 hr after T-cell stimulation, and antigen-induced cytokine production was measured by sandwich ELISA. Recommended pairs of antibodies (coating and detecting) for IL-10 (R&D, Minneapolis, Minn.), INF-γ. Endogen, Woburn, Mass.), IL-13 (PharMingen, (San Diego, Calif.), and TGF-β (Promega, Madison, Wis.) were used according to the manufacturer's instructions. Surface and intracellular staining was performed on Aβ-reactive T-cells after stimulation. All antibodies were purchased from BD Biosciences (San Diego, Calif.) and staining was performed according to the manufacturer's instructions.

Detection of antibody isotype: Wells of 96-well plates are coated with Aβ epitope in bicarbonate coating buffer (pH 9.7) and incubated overnight at 4° C. The wells are then washed and blocked with 3% non-fat dry milk in Tween 20/Tris buffer solution (TTBS) for 1-2 hours at 37° C. After wasting the wells, primary sera from control was added in duplicate at an initial dilution of 1:500 or as indicated and diluted serially in TTBS to 1:64,000. After incubation and washing, anti-human IG conjugated with horseradish peroxidase (HRP) is added as recommended by the manufacturer. Plates are then incubated for 1 hour at 37° C., washed, and freshly prepared IPD substrate solution (o-phenylenediamine in 0.056 M phosphate citrate buffer, pH 5.0; Sigma) is added to develop the reaction. All plates are analyzed spectrophotometrically.

Mice: C57BL6 and SJL mice were purchased from The Jackson Laboratory (Bar Harbor, Me. USA). NOD mice were purchased from Taconic Farms (Germantown, N.Y.). APP Tg J20 line in a C57BL6 background expressing APP under the PDGF promoter were received from Dr. Mucky (Mucke, L., E. Masliah, G. Q. Yu, M. Mallory, E. M. Rockenstein, G. Tatsuno, K. Hu, D. Kholodenko, K. Johnson-Wood, and L. McConlogue. 2000. High-level neuronal expression of abeta 1-42 in wild-type human amyloid protein precursor transgenic mice: synaptotoxicity without plaque formation. J Neurosci 20:4050.). Transgenic SJL mice expressing IFN-γ under the MBP promoter were received from Dr. Owens (Renno, T., V. Taupin, L. Bourbonniere, G. Verge, E. Tran, R. De Simone, M. Krakowski, M. Rodriguez, A. Peterson, and T. Owens. 1998. Interferon-gamma in progression to chronic demyelination and neurological deficit following acute EAE. Mol Cell Neurosci 12:376.). Homozygous IFN-γ-Tg mice were bred with APP-Tg mice to generate double Tg B6SJLF1 mice.

Antigens used in Mice: Aβ1-40 and Aβ1-42 peptides were synthesized in the Biopolymer Laboratory (CND, Brigham and Women's Hospital). Fluorescein isothiocyanate (FITC)-labeled Aβ was purchased from Biosource (Camarillo, Calif.). All other Ab peptide, oligodendrocyte glycoprotein (MOG) 35-55, myelin basic protein (MBP), and proteolipid protein (PLP) 139-15 were synthesized by Quality Controlled Biochemicals (Hopkinton, Mass.). For in vitro stimulation of lymphocytes, Aβ peptides were dissolved in DMSO (Sigma) at 2 mg/ml prior to final dilution in X-vivo media (Bio-Whittaker, Walkersville, Md.). MOG 35-55, MBP, and PLP were dissolved in distilled water at 2 mg/ml. For immunization, Aβ peptides were dissolved in distilled water at 2 mg/ml.

Immunization and measurement of immune responses: Mice were immunized by footpad injection if sacrificed on day 12, for longer periods mice were injected in the flanks. Each mouse received 100 ml of antigen (1 mg/ml), emulsified in an equal volume of complete Freund's adjuvant (CFA). Mice were also injected intravenously with 150 ng pertussis toxin at the time of immunization and 48 hours later. At the indicated time points, mice were bled and popliteal draining lymph nodes (PLN) or spleens were excised and the PLN- or spleen-derived cells were cultured in X-vivo serum-free medium in U-bottom 96-well plates and tested in vitro for antigen-induced proliferation and cytokine production. Antigen-induced cytokine production was measured by sandwich ELISA. Recommended pairs of antibodies (coating and detecting) for IL-2, IL-4, IL-10, and INF-γ were purchased from BD Biosciences (San Diego, Calif.) and used according to the manufacturer's instructions. For proliferation measurements cells were pulsed with 1 mCi 3H-thymidine/well 72 h after stimulation and harvested 12 h later, followed by measuring 3H-thymidine incorporation. Anti-Aβ antibodies in serum were measured by ELISA as described (Monsonego, A., R. Maron, V. Zota, D. J. Selkoe, and H. L. Weiner. 2001. Immune hyporesponsiveness to amyloid beta-peptide in amyloid precursor protein transgenic mice: implications for the pathogenesis and treatment of Alzheimer's disease. Proc Natl Acad Sci USA 98:10273.).

Immunohistochemistry and imaging: Cryosections (6 μm) from brains of APP-Tg+ mice were placed onto glass slides and fixed with ice-cold methanol for 2 min followed by 2% PFA at RT for 4 min. Sections were washed once with distilled water for 5 min and twice with PBS containing 0.05% Tween-20. Sections were then incubated for 1 hour at RT with blocking buffer (PBS containing 3% normal goat serum and 2% bovine serum albumin). The purified primary antibodies rat-anti mouse CD11b and rat-anti mouse CD4 (BD Biosciences), and polyclonal rabbit-anti human Aβ (1282, obtained from the laboratory of Dr. Selkoe) were diluted in blocking buffer 1:100, 1:50, and 1:500, respectively, and added to the sections for 2 hours at RT. For Horseradish Peroxidase (HRP) staining secondary biotinilated antibodies, ABC, and diaminobenzidine (DAB) kits were used (Vector, Burlingame, Calif.), according to manufacture's instructions. Images were taken by a digital camera using the Zeiss imaging system.

For fluorescence staining, Alexa Fluor 488, 594, and 660 were used as secondary antibodies (diluted 1:250 in blocking buffer, 1 hour at RT), and TOTO-3 (diluted 1:1000 in PBS, 5 min at RT) for nuclei staining (Molecular probes). Sections were examined under a Zeiss Laser Scanning Confocal Microscope and 3D analysis software (Zeiss, Thornwood, NY).

Preparation of cultures of mouse brain microglia: Glial cultures were prepared as described (JI). Briefly, cells were dissociated from the cerebral cortex of 1-day-old C57BL/6 mice after careful removal of meninges tissues, and were cultured in poly-D-lysine (PDL)-coated tissue culture flasks in medium supplemented with Dulbecco's modified Eagle's medium (DMEM), 4 mM L-glutamine, 50 U/ml penicillin, 50 mg/ml streptomycin, 10 mM HEPES, 1 mM sodium pyruvate, 10 mM nonessential amino acids, 57.2 mM 2-mercaptoethanol (Sigma, St Louis, Mo.), and 10% fetal calf serum (FCS). On day 7, cultures were incubated with 100 pg/ml IFN-γ for 72 hours and on day 10, the entire glial culture was trypsinized and microglia were labeled with PE-conjugated anti-CD11b and sorted using FACS Vantage SE Cell Sorter.

Aβ uptake and T-cell stimulation by microglia: PE-labeled microglial cells were cultured in MatTek culture dishes (Ashland, Mass.) for 24 hours in the DMEM-FCS medium. FITC-labeled Aβ1-42 (0.1 mg/ml) incubated for 5 hours at 37 C in DMEM-FCS medium was then added to the culture for 30 min and washed twice with DMEM-FCS medium. Live images were then taken for 30 min with 20 sec intervals by a Zeiss Laser Scanning Confocal Microscope and analyzed by 3D analysis software (Zeiss, Thornwood, N.Y.). For the microglia-T-cell coculture experiments, Aβ-reactive T cells were generated as described and labeled with Alexa Flour 488-conjugated anti-mouse CD4. Resting cells were added to the microglia culture together with Aβ1-42.

Monitoring the immune response of a subject: In some cases it is beneficial to monitor the immune response of a patient over time. For example, the relative increase in the relative increase Aβ reactive T-cells. In some instances, an relative increase in Th2 immune response versus a Th1 immune response in a patient can indicate that the patient has a Th2 related condition, e.g., Alzheimer's disease. On the other hand, a relative increase in a Th1 immune response versus a Th2 response can be indicative of a Th1 related disorder such as multiple sclerosis, or alternatively, an encephalitic response.

It is also beneficial to monitor the patient's immune response, such as a T-cell response, before and after the administration of an Aβ vaccine or other treatment. For example, if a patient responds to a vaccine with a relative increase in Th2 immune response versus Th1 immune response, then a health care professional can determine that the vaccine is having the intended immune response, and can use that information to prescribe suitable follow-up treatment, for example a booster of the initial vaccine. However, if the patient responds with a relative increase in Th1 immune response versus Th2 response, than the health care professional can determine that the intended immune response was not achieved and can proscribe adequate necessary treatment, for example, e.g., prescribe an anti-inflammatory.

In some instances, it is desirable to determine the presence of INF-γ in a subject, which can indicate, for example, whether the subject has an infection of the central nervous system. Examples of infections of the central nervous system include, for example, meningitis (bacterial or viral), encephalitis, polymyeloradiculitis, ventriculitis, myelitis, inflammatory polyneuropathy, meninigoencephalitis, acute cerebellar ataxia, aseptic meningitis, transverse myelitis, autonomic neuropathy, primary CNS lymphoma in AIDS, Bartonella henselae, Borrelia burgdorferi, Cryptococcal neoformas, Leptospira interrogans, Mycobacterium pneumoniae, Mycobacterium tuberculosis, Toxoplasma gondii, and Tophryma whippelii.

INF-γ can be measured, for example, from a sample of the cerebral spinal fluid, or serum more generally (e.g., serum from a blood sample of a patient). The INF-γ, can then be detected using standard assays such as an ELISA assay.

The patient's immune response can be measured, for example, using the techniques described above, such as measuring cytokine production or by measuring the presence of antibody in serum.

For example, a patient can provide a blood sample at an initial time where the immune response is determined. This initial determination can then be compared with the same patient's immune response at a second time, e.g., one week, one month, two months, three months, or six months later.

The peptides of this invention can be made by chemical synthesis methods, which are well known, to the ordinarily skilled artisan. See, for example, Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the α-NH2 protected by either t-Boc or F-moc chemistry using side chain protected amino acids on, for example, an Applied Biosystems Peptide Synthesizer Model 430A or 431.

For example, methods of making peptides are well known in the art. One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Alternatively, the longer synthetic peptides can be synthesized by well known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions Appropriate for the selected expression system and host. The peptide is purified and characterized by standard methods.

Pharmaceutical compositions of this invention comprise a peptide described herein or a pharmaceutically acceptable salt thereof; an adjuvant; and in some instances a pharmaceutically acceptable carrier or vehicle. The compositions delineated herein can also include additional therapeutic agents if present, in amounts effective for achieving a modulation of disease or disease symptoms, including disorders relating to Aβ-extracellular plaques or symptoms thereof.

Pharmaceutically acceptable carriers, adjuvants and vehicles that may be used in the pharmaceutical compositions of this invention include, but are not limited to, ion exchangers, alum, alumina, aluminum stearate, lecithin, self-emulsifying drug delivery systems (SEDDS) such as d-a-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

The compositions of the formulae described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation. The methods herein contemplate administration of an effective amount of compound or compound composition to achieve the desired or stated effect. The amount of active ingredient that may be combined with the carrier materials to produce a single dosage form will vary depending upon the host treated and the particular mode of administration.

Specific dosage and treatment regimens for any particular patient will depend upon a variety of factors, including the activity of the specific composition employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered mucosally, such as by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The compositions described herein can be used to treat disorders related to amyloid fibril formation (e.g., an amyloid fibril disorder). An amyloid fibril disorder includes diseases associated with the accumulation of amyloid, which can either be restricted to one organ, “localized amyloidosis”, or spread to several organs, “systemic amyloidosis.” Secondary amyloidosis can be associated with chronic infection (such as tuberculosis) or chronic inflammation (such as rheumatoid arthritis), including a familial form of secondary amyloidosis which is also seen in Familial Mediterranean Fever (FMF) and other types of systemic amyloidosis found in long-term hemodialysis patients. Some examples of disorders related to amyloid fibril formation include the following: Alzheimer's Disease, Down's Syndrome, Dutch Type Hereditary Cerebral Hemorrhage Amyloidosis, Reactive Amyloidosis, Familial Amyloid Nephropathy with Urticaria and Deafness, Muckle-Wells Syndrome, Idiopathic Myeloma; Macroglobulinemia-Associated Myeloma, Familial Amyloid Polyneuropathy, Familial Amyloid Cardiomyopathy, Isolated Cardiac Amyloid, Systemic Senile Amyloidosis, Adult Onset Diabetes, Insulinoma, Isolated Atrial Amyloid, Medullary Carcinoma of the Thyroid, Familial Amyloidosis, Hereditary Cerebral Hemorrhage With Amyloidosis, Familial Amyloidotic Polyneuropathy, Scrapie, Creutzfeldt-Jacob Disease, Gerstmann Straussler-Scheinker Syndrome, Bovine Spongiform Encephalitis, a Prion-mediated disease, and Huntington's Disease.

The invention is further described in the following examples, which do not limit the scope of the invention described in the claims.

### Other Embodiments

A number of embodiments of the invention have been described. Nevertheless, it will be understood that various modifications may be made without departing from the spirit and scope of the invention. Accordingly, other embodiments are within the scope of the following claims.

1. A peptide comprising at least 12 contiguous amino acids of SEQ ID NO:1, excluding amino acids 1-14, wherein the peptide elicits T-cell response upon administration to a subject.

2. The peptide of claim 1 wherein the T-cell response is determined by exposing the peptide to PBMCs.

3. The peptide of claim 1 wherein the T-cell response is in the central nervous system.

4. The peptide of claim 1 wherein the T-cell response is in the brain.

5. The peptide of claim 1 wherein the T-cell response comprises a reduction of Aβ plaques.

6. The peptide of claim 1 wherein the T-cell response reduces Aβ plaques through the clearance of Aβ via microglia or macrophages.

7. The peptide of claim 1 wherein the T-cell response comprises an increase in Th2 Aβ reactive T-cells.

8. The peptide of claim 1 wherein the T-cell response comprises an increase in Th1 Aβ reactive T-cells.

9. The peptide of claim 1 wherein the T-cell response comprises an increase in both Th1 Aβ reactive T-cells and Th2 Aβ reactive T-cells.

10. The peptide of claim 1 wherein the peptide comprises 12 contiguous amino acids from amino acids 15-34 of SEQ ID NO:1.

11. The peptide of claim 1 wherein the peptide comprises amino acids 19-33 of SEQ ID NO:1.

12. The peptide of claim 1 wherein the peptide comprises amino acids 30-42 of SEQ ID NO:1.

13. The peptide of claim 1 comprising a T-cell epitope.

14. The peptide of claim 1 comprising a Th2 epitope.

15. The peptide of claim 1 comprising a Th1 epitope.

16. A composition comprising the peptide of claim 1.

17. The composition of claim 16 wherein the composition comprises an adjuvant.

18. The composition of claim 17 wherein the adjuvant is selected from the group consisting of alum and incomplete Freud's adjuvant.

19. The composition of claim 17 wherein the adjuvant is selected from the group consisting of complete Freud's adjuvant and QS21.

20. A composition comprising a peptide comprising at least 12 contiguous amino acids of SEQ ID NO:1 and an adjuvant, wherein the composition elicits an increase an Th1 Aβ reactive T-cells that is less than ten times as great as an increase in Th2 Aβ reactive T-cells when exposed to PBMCs.

21. The composition of claim 20, wherein the composition elicits a greater increase in Th2 Aβ reactive T-cells than an increase in Th1 Aβ reactive T-cells when exposed to PBMCs.

22. The composition of claim 20, the peptide comprising at least 15 contiguous amino acids of SEQ ID NO:1.

23. The composition of claim 20, the peptide comprising amino acids 15-30 of SEQ ID NO:1.

24. The composition of claim 20, the peptide comprising amino acids 16-31 of SEQ ID NO:1.

25. The composition of claim 20, the peptide comprising amino acids 18-33 of SEQ ID NO:1.

26. The composition of claim 20, the peptide comprising amino acids 30-42 of SEQ ID NO:1.

27. The composition of claim 20, the peptide comprising a Th2 epitope.

28. The composition of claim 20, the peptide comprising a Th1 epitope.

29. The composition of claim 20, the peptide not comprising amino acids 1-14 of SEQ ID NO:1.

30. The composition of claim 20, the adjuvant comprising alum or incomplete Freud's adjuvant.

31. The composition of claim 20, further comprising a pharmaceutically acceptable carrier.

32. The composition of claim 20, wherein the peptide comprises amino acids 18-33 of SEQ ID NO:1, and the adjuvant comprises alum.

33. The composition of claim 20, wherein the amount of Th2 Aβ reactive T-cells are determined by expression of IL-4, IL-5, IL-10, or IL-13, and the amount Th1 Aβ reactive T-cells are determined by expression of IFN-γ, IL-12, or TNF-α.

34. The composition of claim 20, wherein the amount of Th2 Aβ reactive T-cells are determined by the expression of IgG1 or IgG4 and the amount Th1 Aβ reactive T-cells are determined by expression of IgG2 or IgG3.

35. The composition of claim 20, wherein the peptide elicits a greater increase in Th2 Aβ reactive T-cells than increase in Th1 Aβ reactive T-cells in the absence of adjuvant.

36. The composition of claim 20, wherein the adjuvant elicits a greater increase in Th2 Aβ reactive T-cells than increase in Th1 Aβ reactive T-cells in the absence of peptide.

37. The composition of claim 20, wherein both the peptide and the adjuvant independently a greater increase in Th2 Aβ reactive T-cells than increase in Th1 Aβ reactive T-cells.

38. A method of eliciting a T-cell immune response in a patient, the method comprising administering to the patient a peptide comprising at least 12 contiguous amino acids of SEQ ID NO:1, excluding amino acids 1-14.

39. The method of claim 38 wherein the peptide is administered parenterally.

40. The method of claim 38 wherein the peptide is administered mucosally.

41. The method of claim 38 wherein the immune response comprises a reduction of Aβ plaques in the central nervous system.

42. The method of claim 38 wherein the immune response comprises a reduction of Aβ plaques in the brain.

43. The method of claim 38 wherein the reduction of Aβ plaques occurs by the clearance of Aβ via microglia or macrophages.

44. A method for eliciting a T-cell immune response in a patient, the method comprising administering to the patient a composition comprising a peptide comprising at least 12 contiguous amino acids of SEQ ID NO:1, excluding amino acids 1-14.

45. The method of claim 44, wherein the composition is administered parenterally.

46. The method of claim 44, wherein the composition is administered mucosally.

47. The method of claim 44, wherein the composition is administered nasally.

48. The method of claim 44, wherein the administration of the composition elicits an increase in Th2 Aβ reactive T-cells in the patient.

49. The method of claim 44, wherein the administration of the composition elicits an increase in Th1 Aβ reactive T-cells in the patient.

50. The method of claim 44, wherein the administration of the composition elicits an increase in Th1 Aβ reactive T-cells and an increase in Th2 Aβ reactive T-cells in the patient.

51. A method for eliciting an immune response in a patient, the method comprising administering to the patient a composition comprising at least 12 contiguous amino acids of SEQ ID NO:1 and an adjuvant, wherein the composition elicits a greater increase in Th2 Aβ reactive T-cells than an increase in Th1 Aβ reactive T-cells when exposed to PBMCs.

52. The method of claim 51 wherein the composition is administered mucosally.

53. A method of treating a patient having an amyloid fibril disorder, the method comprising administering to the patient the peptide of claim 1.

54. The method of claim 53, wherein the amyloid fibril disorder is dementia.

55. The method of claim 54, wherein the dementia is Alzheimer's disease.

56. The method of claim 53, wherein the amyloid fibril disorder is Down's Syndrome, Dutch Type Hereditary Cerebral Hemorrhage Amyloidosis, Reactive Amyloidosis, Familial Mediterranean Fever, Familial Amyloid Nephropathy with Urticaria and Deafness, Muckle-Wells Syndrome, Idiopathic Myeloma; Macroglobulinemia-Associated Myeloma, Familial Amyloid Polyneuropathy, Familial Amyloid Cardiomyopathy, Isolated Cardiac Amyloid, Systemic Senile Amyloidosis, Adult Onset Diabetes, Insulinoma, Isolated Atrial Amyloid, Medullary Carcinoma of the Thyroid, Familial Amyloidosis, Hereditary Cerebral Hemorrhage with Amyloidosis, Familial Amyloidotic Polyneuropathy, Scrapie, Creutzfeldt-Jacob Disease, Gerstmann Straussler-Scheinker Syndrome, Bovine Spongiform Encephalitis, a Prion-mediated disease, or Huntington's Disease.

57. A method of treating a patient having an amyloid fibril disorder, the method comprising administering to the patient a composition of claim 20.

58. The method of claim 57, wherein the amyloid fibril disorder is dementia.

59. The method of claim 58, wherein the dementia is Alzheimer's disease.

60. The method of claim 57, wherein the amyloid fibril disorder is Down's Syndrome, Dutch Type Hereditary Cerebral Hemorrhage Amyloidosis, Reactive Amyloidosis, Familial Mediterranean Fever, Familial Amyloid Nephropathy with Urticaria and Deafness, Muckle-Wells Syndrome, Idiopathic Myeloma; Macroglobulinemia-Associated Myeloma, Familial Amyloid Polyneuropathy, Familial Amyloid Cardiomyopathy, Isolated Cardiac Amyloid, Systemic Senile Amyloidosis, Adult Onset Diabetes, Insulinoma, Isolated Atrial Amyloid, Medullary Carcinoma of the Thyroid, Familial Amyloidosis, Hereditary Cerebral Hemorrhage with Amyloidosis, Familial Amyloidotic Polyneuropathy, Scrapie, Creutzfeldt-Jacob Disease, Gerstmann Straussler-Scheinker Syndrome, Bovine Spongiform Encephalitis, a Prion-mediated disease, or Huntington's Disease.

61. The method of claim 57, wherein the composition is administered parenterally.

62. The method of claim 57, wherein the composition is administered mucosally.

63. The method of claim 57, wherein the composition is administered nasally.

64. A method of immunizing a patient, the method comprising administering to the patient a peptide of claim 1.

65. A method of immunizing a patient, the method comprising administering to the patient a composition of claim 20.

66. The method of claim 65, wherein the composition is administered orally.

67. The method of claim 65, wherein the composition is administered parenterally.

68. The method of claim 65, wherein the composition is administered mucosally.

69. The method of claim 65, wherein the composition is administered nasally.

70. A method for immunizing a patient comprising:
a) identifying a patient as free of infection in the central nervous system; and
b) administering to the identified patient a composition comprising a peptide comprising at least 12 contiguous amino acids of SEQ ID NO:1.

71. The method of claim 70 wherein the patient is identified as free of infection in the central nervous system by being substantially free of INF-γ in the central nervous system.

72. The method of claim 70 wherein the cerebral spinal fluid is substantially free of INF-γ.

73. The method of claim 70 wherein the INF-γ is measured using an ELISA assay.

74. The method of claim 70 comprising
c) isolating PBMCs from a patient;
d) exposing the PBMCs to a library of Aβ peptides; and
e) identifying an Aβ peptide within the library that elicits a greater increase in Th2 Aβ reactive T-cells than an increase in Th1 Aβ reactive T-cells when exposed to PBMCs.

75. A method of identifying a peptide for eliciting an immune response in an individual, the method comprising;
a) isolating PBMCs from a patient;
b) exposing the PBMCs to a library of Aβ peptides; and
c) identifying an Aβ peptide within the library that elicits a T-cell immune response when exposed to PBMCs.

76. The method of claim 75, wherein the library of Aβ peptides comprises peptides comprising at least 10 contiguous amino acids of SEQ ID NO:1.

77. The method of claim 75, wherein the T-cell response comprises an increase in Th1 Aβ reactive T-cells or an increase in Th2 Aβ reactive T-cells.

78. The method of claim 75, wherein the T-cell response comprises an increase in Th1 Aβ reactive T-cells and an increase in Th2 Aβ reactive T-cells.

79. The method of claim 75, wherein the an Aβ peptide elicits a greater increase in Th2 Aβ reactive T-cells than an increase in Th1 Aβ reactive T-cells.

80. The method of claim 75, wherein the amount of Th2 Aβ reactive T-cells are determined by expression of IL-4, IL-5, IL-10, or IL-13, and the amount Th1 Aβ reactive T-cells are determined by expression of IFN-γ, IL-12, or TNF-α.

81. The method of claim 75, wherein the amount of Th2 Aβ reactive T-cells are determined by the expression of IgG1 or IgG4 and the amount Th1 Aβ reactive T-cells are determined by expression of IgG2 or IgG3.

82. The method of claim 75, wherein the Aβ peptides are covalently attached to an MHC class II molecule.

83. The method of claim 75, wherein the Aβ peptides are non-covalently attached to an MHC class II molecule.

84. A method of immunizing an individual, the method comprising;
a) isolating PBMCs from an individual;
b) exposing the PBMCs to a library of Aβ peptides;
c) identifying an Aβ peptide within the library that elicits a T-cell response; and
d) administering to the individual the identified Aβ peptide, thereby immunizing the individual.

85. The method of claim 84, wherein the library of Aβ peptides comprises peptides comprising at least 10 contiguous amino acids of SEQ ID NO:1.

86. The method of claim 84, wherein the T-cell response comprises an increase in Th1 Aβ reactive T-cells or an increase in Th2 Aβ reactive T-cells.

87. The method of claim 84, wherein the T-cell response comprises an increase in Th1 Aβ reactive T-cells and an increase in Th2 Aβ reactive T-cells.

88. The method of claim 84, further comprising identifying an Aβ peptide within the library that elicits a greater increase in Th2 Aβ reactive T-cells than an increase in Th1 Aβ reactive T-cells when exposed to PBMCs.

89. The method of claim 84, further comprising determining the relative amounts of Th2 Aβ reactive T-cells and Th1 Aβ reactive T-cells at a time prior to administering to the individual the identified Aβ peptide, t₀, and determining the relative amounts of Th2 Aβ reactive T-cells and Th1 Aβ reactive T-cells at a time after administering to the individual the identified Aβ peptide, t₁.

90. The method of claim 84, wherein the amount of Th2 Aβ reactive T-cells are determined by expression of IL-4, IL-5, IL-10, or IL-13, and the amount Th1 Aβ reactive T-cells are determined by expression of IFN-γ, IL-12, or TNF-α.

91. The method of claim 84, wherein the amount of Th2 Aβ reactive T-cells are determined by the expression of IgG1 or IgG4 and the amount Th1 Aβ reactive T-cells are determined by expression of IgG2 or IgG3.

92. The method of claim 84, wherein the Aβ peptides are covalently attached to an MHC class II molecule.

93. The method of claim 84, wherein the Aβ peptides are non-covalently attached onto an MHC class II molecule.

94. A method of identifying a candidate composition for eliciting an immune response in an individual, the method comprising;
a) isolating PBMCs from a patient;
b) exposing the PBMCs to a library of compositions, each composition comprising an Aβ peptide comprising at least 10 contiguous amino acids of SEQ ID NO:1 and an adjuvant; and
c) identifying as the candidate composition a composition within the library of compositions that elicits a T-cell response.

95. The method of claim 94, wherein the T-cell response comprises an increase in Th2 Aβ reactive T-cells.

96. The method of claim 94, wherein the T-cell response comprises an increase in Th1 Aβ reactive T-cells.

97. The method of claim 94, wherein the T-cell response comprises an increase in both Th1 Aβ reactive T-cells and Th2 Aβ reactive T-cells.

98. The method of claim 94, wherein the composition elicits a greater increase in Th2 Aβ reactive T-cells than an increase in Th1 Aβ reactive T-cells when exposed to PBMCs.

99. The method of claim 94, wherein the amount of Th2 Aβ reactive T-cells are determined by expression of IL-4, IL-5, IL-10, or IL-13, and the amount Th1 Aβ reactive T-cells are determined by expression of IFN-γ, IL-12, or TNF-α.

100. The method of claim 94, wherein the amount of Th2 Aβ reactive T-cells are determined by the expression of IgG1 or IgG4 and the amount Th1 Aβ reactive T-cells are determined by expression of IgG2 or IgG3.

101. The method of claim 94, wherein the Aβ peptides are covalently attached to an MHC class II molecule.

102. The method of claim 94, wherein the Aβ peptides are non-covalently attached to an MHC class II molecule.

103. A method of immunizing an individual, the method comprising;
a) isolating PBMCs from a patient;
b) exposing the PBMCs to a library of compositions, each composition comprising an Aβ peptide comprising at least 10 contiguous amino acids of SEQ ID NO:1 and an adjuvant;
c) identifying a composition within the library that elicits a T-cell response; and
d) administering the identified composition to the individual, thereby immunizing the individual.

104. The method of claim 103, wherein the T-cell response comprises an increase in Th2 Aβ reactive T-cells.

105. The method of claim 103, wherein the T-cell response comprises an increase in Th1 Aβ reactive T-cells.

106. The method of claim 103, wherein the T-cell response comprises an increase in both Th1 Aβ reactive T-cells and Th2 Aβ reactive T-cells.

107. The method of claim 103, wherein the composition reduces Aβ plaques in the central nervous system.

108. The method of claim 103, wherein the Aβ plaques are reduced by Aβ clearance via microglia and macrophages.

109. The method of claim 103, wherein the composition elicits a greater increase in Th2 Aβ reactive T-cells than an increase in Th1 Aβ reactive T-cells when exposed to PBMCs.

110. The method of claim 103, further comprising; determining the relative amounts of Th2 Aβ reactive T-cells and Th1 Aβ reactive T-cells at a time prior to administering to the individual the identified Aβ peptide, t₀, and determining the relative amounts of Th2 Aβ reactive T-cells and Th1 Aβ reactive T-cells at a time after administering to the individual the identified Aβ peptide, t₁.

111. The method of claim 103, wherein the amount of Th2 Aβ reactive T-cells are determined by expression of IL-4, IL-5, IL-10, or IL-13, and the amount Th1 Aβ reactive T-cells are determined by expression of IFN-γ, IL-12, or TNF-γ.

112. The method of claim 103, wherein the amount of Th2 Aβ reactive T-cells are determined by the expression of IgG1 or IgG4 and the amount Th1 Aβ reactive T-cells are determined by expression of IgG2 or IgG3.

113. The method of claim 103, wherein the Aβ peptides are adsorbed onto an MHC class II molecule.

114. A method for determining an adverse reaction to an immunization with an Aβ antigen in a patient, the method comprising;
a) measuring the relative amounts of Th2 Aβ reactive T-cells and Th1 Aβ reactive T-cells in a patient at an initial time prior to immunization, t₀;
b) immunizing the patient with an Aβ antigen;
c) measuring the relative amounts of Th2 Aβ reactive T-cells and Th1 Aβ reactive T-cells in a patient at a time subsequent to immunization, t₁; and
d) comparing the relative responses of Th2 Aβ reactive T-cells and Th1 Aβ reactive T-cells at to and t₁, wherein an increase in relative amounts of Th1 Aβ reactive T-cells to relative amounts of Th2 Aβ reactive T-cells from t₀ to t₁ indicates an adverse reaction to the immunization.

115. A method for determining an adverse reaction to an immunization with an Aβ antigen in a patient, the method comprising;
a) measuring an amount of Th1 Aβ reactive T-cells and an amount of Th2 Aβ reactive T-cells in a patient at an initial time prior to immunization, t₀;
b) immunizing the patient with an Aβ antigen;
c) measuring an amount of Th1 Aβ reactive T-cells and an amount of Th2 Aβ reactive T-cells in a patient at a time subsequent to immunization, t₁;
d) determining whether the patient experienced an increase in Th1 Aβ reactive T-cells that is more than ten times greater than the increase in Th2 Aβ reactive T-cells from t₀ to t₁ and whether IFN-γ is present in the central nervous system, wherein an relative increase in Th1 Aβ reactive T-cells greater than ten times the increase in Th2 Aβ reactive T-cells from t₀ to t₁ combined with the presence of INF-γ in the central nervous system indicates an adverse reaction.

116. A method of diagnosing an Aβ fibril disorder in a patient, the method comprising;
a) measuring the amount of Aβ reactive T-cells in a patient;
b) comparing the amount Aβ reactive T-cells to a standard; and
c) diagnosing the patient with an Aβ fibril disorder if the amount of Aβ reactive T-cells in the patient as determined in step b) is higher than the standard.


SEQUENCE LISTING (2 sequences)

SEQ ID NO: 1 - protein, 42 aa, Homo sapiens [sequence omitted]

SEQ ID NO: 2 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN